(19) United States
(12) Patent Application Publication
(10) Pub. No.: US 2024/0055100 A1
(43) Pub. Date: Feb. 15, 2024

(54) MACHINE LEARNING SOLUTION TO PREDICT PROTEIN CHARACTERISTICS

(71) Applicant: MICROSOFT TECHNOLOGY LICENSING, LLC, Redmond, WA (US)

(72) Inventors: Sara Malvar MAUA, Sao Paulo (BR); Anvita Kriti Prakash BHAGAVATHULA, Providence, RI (US); Ranveer CHANDRA, Kirkland, WA (US); Maria Angels de LUIS BALAGUER, Raleigh, NC (US); Anirudh BADAM, Issaquah, WA (US); Roberto DE MOURA ESTEVÃO FILHO, Raleigh, NC (US); Swati SHARMA, Hayward, CA (US)

(21) Appl. No.: 18/146,123

(22) Filed: Dec. 23, 2022

Related U.S. Application Data
(60) Provisional application No. 63/371,508, filed on Aug. 15, 2022.

(51) Int. Cl. G16H 20/60

(52) U.S. Cl. CPC G16H 20/60

(57) ABSTRACT

This disclosure provides a machine learning technique to predict a protein characteristic. A first training set is created that includes, for multiple proteins, a target feature, protein sequences, and other information about the proteins. A first machine learning model is trained and then used to identify which of the features are relevant as determined by feature importance or causal relationships to the target feature. A second training set is created with only the relevant features. Embeddings generated from the protein sequences are also added to the second training set. The second training set is used to train a second machine learning model. The first and second machine learning models may be any type of regressors. Once trained, the second machine learning model is used to predict a value for the target feature for an uncharacterized protein. The model of this disclosure provides 91% accuracy in predicting an ideal digestibility score.

### PRIORITY APPLICATION

This application claims the benefit of and priority to Provisional Application No. 63/371,508, filed Aug. 15, 2022, the entire contents of which are incorporated herein by reference.

### BACKGROUND

As the world's population increases rapidly and because land, water, and food resources are limited, it is becoming increasingly important to provide quality protein to meet human nutritional needs. A sufficient dietary supply of protein is necessary to support the health and well-being of human populations. New foods and alternative proteins are created as a solution to this need. However, the current techniques for evaluating the characteristics of new food proteins are labor intensive and generally require human or animal test subjects.

Alternative techniques for rapidly evaluating characteristics of proteins would have great utility in developing new food items and alternative protein sources. This disclosure is made with respect to these and other considerations.

### SUMMARY

This disclosure provides a data-driven application using machine learning to achieve a faster and less expensive technique for predicting protein characteristics. An initial training set is created with data from proteins for which the value of a target feature, or label, is known. This target feature is the feature that the machine learning model is trained to predict and may be any characteristic of a protein such as digestibility, flavor, or texture. The training set also includes multiple other features for the proteins such as nutritional data of a food item containing the protein and physiochemical features determined from the protein sequence.

A machine learning model is trained with this labeled dataset. The machine learning model may be any type of machine learning model that can learn a non-linear relationship between a dependent variable and multiple independent variables. For example, the machine learning model may be a regressor. This machine learning model is used to identify which features from the initial training set are most relevant for predicting the target feature. The relevant features are identified by one or both of feature importance and causal inference. The relevant features are a subset of all the features used to train the machine learning model. There may be, for example, hundreds of features in the initial training set, but only tens of features in the smaller subset of relevant features.

This smaller subset of relevant features and the target feature are then used to create a second training set. Embeddings generated from the protein sequences are also added to this second training set. Protein sequences are ordered strings of information, the series of amino acids in the protein, and any one of multiple techniques can be used to create the embeddings. For example, a technique originally developed for natural language processing called the transformer model that uses multi-headed attention can create embeddings from protein sequences.

The second training set, with the smaller subset of features and embeddings, is used to train a second machine learning model. This second machine learning model may be the same or different type of model than the machine learning model used earlier. Once trained, the second machine learning model can be used to predict a value of the target feature for uncharacterized proteins. The techniques of this disclosure use machine learning to predict protein characteristics with no animal testing or costly experiments and surprisingly high accuracy. The predictions may be used to guide further experimental analysis. These techniques are also flexible and can be used for any protein feature for which there is labeled training data.

Features and technical benefits other than those explicitly described above will be apparent from a reading of the following Detailed Description and a review of the associated drawings. This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter. The term “techniques,” for instance, may refer to system(s), method(s), computer-readable instructions, module(s), algorithms, hardware logic, and/or operation(s) as permitted by the context described above and throughout the document.

### BRIEF DESCRIPTION OF THE DRAWINGS

The Detailed Description is described with reference to the accompanying figures. In the figures, the left-most digit(s) of a reference number identifies the figure in which the reference number first appears. The same reference numbers in different figures indicate similar or identical items. References made to individual items of a plurality of items can use a reference number with a letter of a sequence of letters to refer to each individual item. Generic references to the items may use the specific reference number without the sequence of letters.

FIG. 1 shows conventional techniques for determining the digestibility score of a food item in comparison to a machine learning technique.

FIGS. 2A and 2B show an illustrative architecture for training a machine learning model with a target feature of a protein, information about that protein, and other features derived from the protein sequence.

FIG. 3 shows an illustrative architecture for using a trained machine learning model to predict a value for a target feature of an uncharacterized protein.

FIG. 4 is a flow diagram of an illustrative method for creating a training set and training a machine learning model with the training set.

FIG. 5 is a flow diagram of an illustrative method for using a trained machine learning model to predict a value for a target feature of an uncharacterized protein.

FIG. 6 is a computer architecture diagram illustrating a computing device architecture for a computing device capable of implementing aspects of the techniques and technologies presented herein.

FIG. 7 is a diagram illustrating a distributed computing environment capable of implementing aspects of the techniques and technologies presented herein.

### DETAILED DESCRIPTION

New and alternative proteins are created to meet specific nutritional needs such as in infant formula, to provide meatless food alternatives, and for numerous other reasons. Some of the key characteristics of food proteins are digestibility, texture, and flavor. Characterization of proteins that have not previously been used in food can also be necessary to obtain regulatory approval. However, the testing required to experimentally determine this information is expensive and time consuming. At present there is no data driven or machine learning approach to characterize protein features such as digestibility, texture, and flavor. The inventors have identified a way of creating machine learning models through selection of training attributes, feature reduction, and creation of a curated dataset that results in surprisingly high accuracy of predictions for target characteristics of uncharacterized proteins and food items.

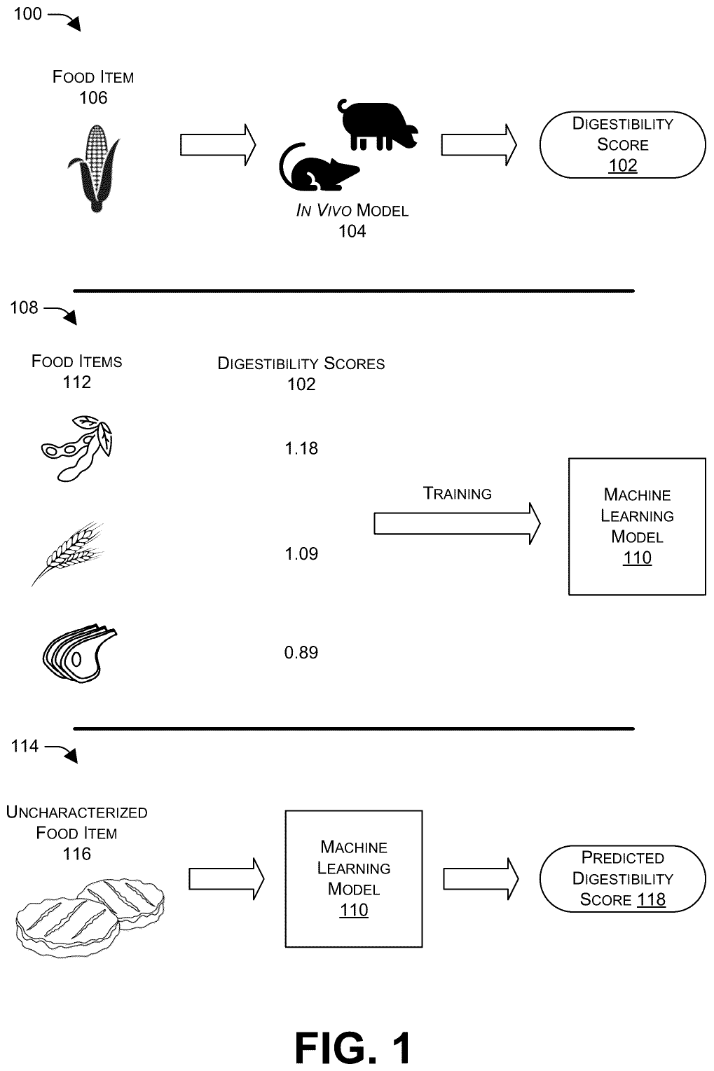

FIG. 1 compares conventional techniques for determining the protein digestibility of proteins in a food item 106 with the machine learning techniques of this disclosure. The top frame 100 of FIG. 1 illustrates conventional techniques for experimentally determining protein digestibility of proteins in a food item 106. Protein digestibility refers to how well a given protein is digested. Protein digestibility can be represented by a digestibility score 102. There are multiple known ways to determine a digestibility score 102 including PDCAAS (Protein Digestibility Corrected Amino Acid Score) and DIAAS (Digestible Indispensable Amino Acid Score). Both PDCAAS and DIAAS are used to evaluate the quality of a protein as a nutritional source for humans.

One existing technique to determine a digestibility score 102 uses an in vivo model 104 (e.g., rat, pig, or human) that takes 3-4 days to fully characterize a food item 106. Calculating a DIAAS score requires experimentally determining ileal digestibility by surgical implantation of a post-valve T-caecum cannula in pigs or use of a naso-ileal tube in humans. Fecal digestibility is used to calculate PDCAAS and is typically done by analysis of rat fecal matter. In vitro characterization using enzymes is an alternative, but this takes 1-2 days with overnight incubation and does not have 100% correlation with in vivo experiments.

The middle frame 108 of FIG. 1 shows an overview of a technique for training a machine learning model 110 to learn the relationship between food items 112 and their corresponding digestibility scores 102 of proteins in those food items. The digestibility scores 102 are scores calculated through conventional experimental techniques. The digestibility scores 102 may be PDCAAS or DIAAS scores. Alternatively, the digestibility scores 102 can be an intermediate score used to calculate PDCASS or DIAAS such as ileal digestibility or fecal digestibility.

Information about the food items 112 used for training the machine learning model 110 includes the protein sequence of proteins from one or more protein families and other information known about the food items 112. Numerous other features of the proteins, such as amino acid composition, can be derived from the protein sequence. Additional information about the food items 112 used to train the machine learning model 110 can include nutritional information such as fat, potassium, and sodium content. Categorial variables related to the food type of the food items 112 (e.g., processed food, dairy, meat, etc.) may also be used for training. Information about the food items 112 that may be included in the training data can describe the preparation and/or storage of the food item as well as antinutritional factors and farming practices. For example, information about preparation may indicate if the food item was consumed raw or cooked. If cooked, details about the cooking such as time and temperature could be included. Similarly, storage information may describe if the food item was fresh, refrigerated, frozen, freeze-dried, and could indicate the length of storage. Antinutritional factors can indicate the presence of things known to decrease protein digestibility such tannins or polyphenols or protease inhibitors that can inhibit trypsin, pepsin, and other proteases from breaking down proteins.

Thus, multiple types of information are collected for food items 112 for which digestibility scores 102 are known to create a set of labeled training data. The labeled training data is used for supervised machine learning. The machine learning model learns non-linear relationships between the values of the digestibility scores 102 and the other features that characterize the food items 112. Any suitable type of machine learning model that can estimate the relationship between a dependent variable and one or more independent variables may be used.

The bottom frame 114 of FIG. 1 illustrates use of the machine learning model 110 after training. Information about an uncharacterized food item 116 is provided to the machine learning model 110. That information includes the protein sequence or at least one protein in the uncharacterized food item 116 and other information that cannot be derived from the protein sequence such as nutritional information. The information about the uncharacterized food item 116 that is provided to the machine learning model 110 may be the same information that was used to train the machine learning model 110. A selected subset of the information (e.g., identified through feature reduction) used to train the machine learning model 110 may also be used.

The machine learning model 110 produces a predicted digestibility score 118 based on the relationships learned during training. The predicted digestibility score 118 may in turn be used to calculate another characteristic of the uncharacterized protein 116. For example, an ileal or fecal digestibility score may then be used to calculate a PDCAAS or DIAAS scores with conventional techniques. The predicted digestibility score 118 for a food items represents the digestibility scores for each of the identified proteins in the food item.

The machine learning model 110 created and trained by the inventors as described in greater detail below, predicts the correct ileal digestibility coefficient with a surprisingly high 91% accuracy. Even though FIG. 1 shows digestibility scores the techniques of this disclosure have broader applicability and can be adapted to predict any feature or characteristic of proteins for which there is suitable training data.

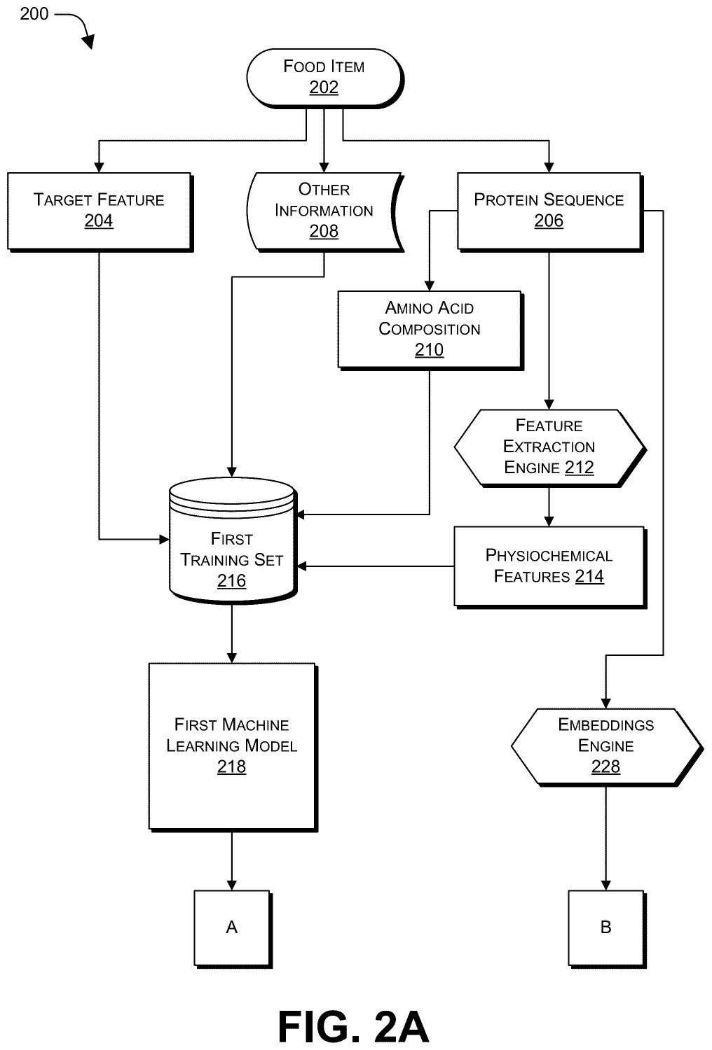

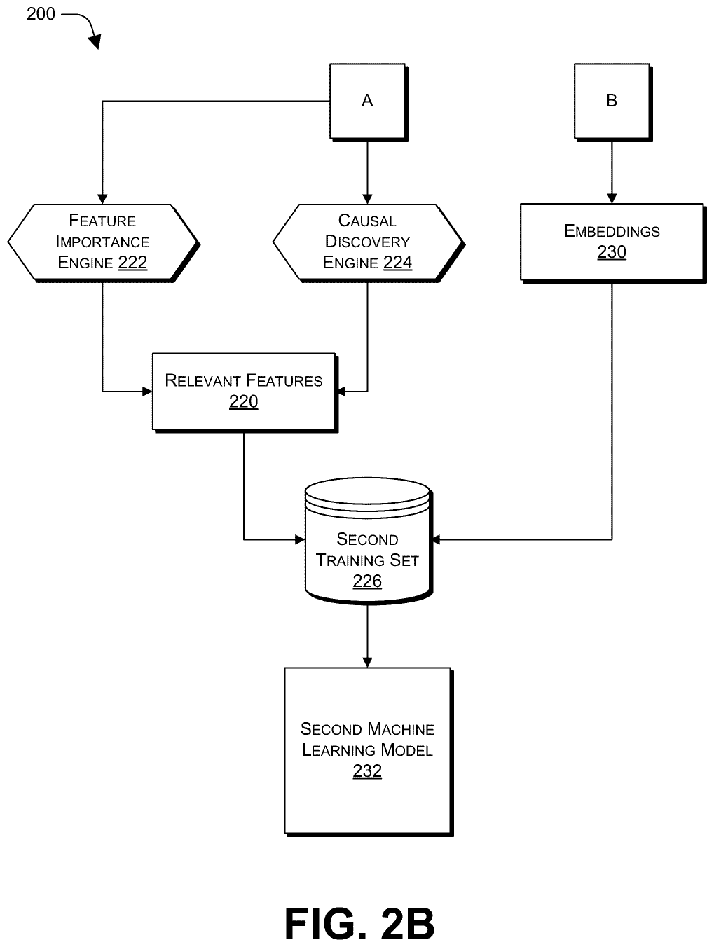

FIGS. 2A and 2B show an architecture 200 for training machine learning models with protein features. The inventors have identified a way of creating machine learning models through selection of training attributes, feature reduction, and creation of a curated dataset that results in surprisingly high accuracy of predictions for target characteristics of uncharacterized proteins. The architecture 200 illustrates training using a single food item 202 for simplicity. However, in practice this technique uses information from many food items to fully train the machine learning models. Families of proteins present in the food item 202 are known and may be identified. One, two, three, or more protein families can be identified for each food item 202. Examples of protein families include albumins, caseins, and globulins. Training a machine learning model on features of a food item 202 may include training on one or more proteins from each of the identified protein families in the food item. Existing databases contain data on proteins in food items including the protein families and amounts of proteins.

The food item 202 is a food item 202 for which labeled training data exists. The label in the training data is referred to as a target feature 204. This is the feature of proteins that the machine learning models are trained to predict. The target feature 204 may be any characteristic of the food item 202 such as, but not limited to, digestibility, texture, or flavor. For example, the target feature 204 may be the ileal digestibility score or the fecal digestibility score. The target feature 204 may be known through existing databases or published literature. The target feature 204 may also be determined experimentally such as, for example, through convention techniques for determining ileal digestibility or fecal digestibility.

The food item 202 is identified by at least on protein sequence 206. Multiple protein sequences 206 may be used to describe a food item 202. A protein sequence is the series of amino acids for that protein. Protein sequences may be represented as strings of values such as a series of single-letter codes, three-letter codes, or numeric representations of each protein. The sequences of many proteins are known and can be accessed from existing databases. The protein sequence 206 can also be determined from the deoxyribose nucleic acid (DNA) sequence of the coding region of a gene for the food item 202. Protein sequences may also be determined through protein sequencing. The sequences of unknown and newly-discovered proteins may be identified through protein sequencing.

Other information 208 is also obtained for the food item 202. Other information 208 includes any other type of information about the food item 202 other than the protein sequence 206 and the target feature 204. Other information 208 may be found in exiting databases or records that describe features of the food item 202. One example of other information 208 for food proteins is nutritional information. Categorical information such as category of the food item 202 or a protein category of the protein sequence 202 may also be included in the other information 208

One type of other information for a food item is nutritional information. Nutritional information provides information about nutrients in the food item 202. A nutrient is a substance used by an organism to survive, grow, and reproduce. A database with nutritional information may contain information on vitamins, minerals, calories, fiber, and the like. Nutritional information may also include indispensable amino acid breakdown profiles. An indispensable amino acid, or essential amino acid, is an amino acid that cannot be synthesized from scratch by the organism fast enough to supply its demand and must therefore come from the diet. The nine essential amino acids for humans are: histidine, isoleucine, leucine, lysine, methionine, phenylalanine, threonine, tryptophan, and valine.

Another example of other information 208 that may be obtained for food items 202 is processing. Processing is related to how the food item 202 is prepared. For instance, protein digestibility is affected by heat so cooking techniques such as temperature and time may be used as features included in the other information 208 that is incorporated into the machine learning models.

The protein sequence 206 is used to derive other features of the food item 202. For example, an amino acid composition 210 can be derived from the protein sequence 206. The amino acid composition 210 is the number, type, and ratios of amino acids present in a protein. The amino acid composition 210 determines the native structure, functionality, and nutritional quality of a protein in a set environment.

A feature extraction engine 212 is used to extract physiochemical features 214 from the protein sequence 206. Examples of physiochemical features 214 include the amount of nitrogen, the amount of carbon, hydrophobicity value of the food item 202 and the like. Some of the features also represent aspects of the secondary protein structure. The features may be represented as vectors. Many techniques are known to those of ordinary skill in the art for determining physicochemical features 214 from the protein sequence 206.

One example tool that may be used as the feature extraction engine 212 is Protlearn. Protlearn is a feature extraction tool for protein sequences that allows the user to extract amino acid sequence features from proteins or peptides, which can then be used for a variety of downstream machine learning tasks. The feature extraction engine 212 can then be used to compute amino acid sequence features from the dataset, such as amino acid composition or AAIndex-based physicochemical properties. The AAIndex, or the Amino Acid Index Database, is a collection of published indices that represent different physicochemical and biological properties of amino acids. The indices for a protein are calculated by averaging the index values for all of the individual amino acids in the protein sequence 206. Protlearn can provide multiple physiochemical features 214 including: length, amino acid composition, AAIndex1-based physicochemical properties, N-gram composition (computes the di- or tripeptide composition of amino acid sequences), Shannon entropy, fraction of carbon atoms, fraction of hydrogen atoms, fraction of nitrogen atoms, fraction of oxygen atoms, fraction of sulfur atoms, Position-specific amino acids, Sequence motifs, Atomic and bond composition, total number of bonds, number of single bonds, number of double bonds, Binary profile pattern, Composition of k-spaced amino acid pairs, Conjoint triad descriptors, Composition/Transition/Distribution—Composition, Composition/Transition/Distribution—Transition, Composition/Transition/Distribution—Distribution, Normalized Moreau-Broto autocorrelation based on AAIndex1, Moran's I based on AAIndex1, Geary's C based on AAIndex1, Pseudo amino acid composition, Amphiphilic pseudo amino acid composition, Sequence-order-coupling number, and Quasi-sequence-order. Techniques for determining these physiochemical features are known to those of ordinary skill in the art and described in on the World Wide Web at protlearn.readthedocs.io/en/latest/feature_extraction.html. Protlearn may al so be used to extract features from the AAindex. However, other existing or newly developed tools besides Protlearn can be used to extract features from protein sequences.

The features of the food item 202 are combined to create a first training set 216. This first training set 216 includes the target feature 204, other information 208 (e.g., nutritional information), the amino acid composition 210, and the physiochemical features 214. In some implementations, the protein sequence 206 itself is not included in the first training set 216 but used only as the source of other features. In the first training set 216, each entry is labeled with the target feature 204 and has potentially a very large number of other pieces of information such as nutritional information and the features extracted from the protein sequence 206. Thus, the first training set 216 could potentially include a very large number of features such as hundreds or thousands of features. In one implementation for predicting ileal digestibility values, the first training set 216 contains 1671 features from 189 food items.

The data used to create the first training set 216 may come from multiple sources such as public or private databases. The databases or data sources used will depend on the protein characteristic that is modeled. If the various types of data come from different sources, they can be merged and joined into a single dataset suitable for training a machine learning model. One example of a public database that may be used is UniProt. UniProt is a freely accessible database of protein sequence and functional information with many entries derived from genome sequencing projects. It contains a large amount of information about the biological function of proteins derived from the research literature including the structure and the sequence of proteins.

The first training set 216 is used to train a first machine learning model 218. The first machine learning model 218 attempts to determine the strength and character of the relationship between the target feature 204 and other features provided from the other information 208 and derived from the protein sequence 206. The first machine learning model 218 can be used to find linear or non-linear relationships. The first machine learning model 218 may use the statistical modeling technique of regression analysis. The first machine learning model 218 can be any type of machine learning model. For example, the first machine learning model 218 may be a decision tree or a regressor such as a random forest regressor.

In some implementations, the first machine learning model 218 is based on boosting and bagging techniques of decision trees such as XGBoost or LightGBM. XGBoost is an ensemble approach with a gradient descent—boosted decision tree algorithm. LightGBM is an improvement framework based on the gradient descent—boosted decision tree algorithm and is more powerful than the previous XGBoost with a fast training speed and less memory occupation. One technique for optimizing hyperparameters of these three models is a combination of a randomized grid search technique and manual tuning using stratified 5-fold cross-validation on the first training set 216.

The first machine learning model 218 is trained to predict the target feature 204 for a food item given the same types of information about that food item that were used to create the first training set 216. While this first machine learning model 218 has predictive ability, it is improved as described below.

Following path “A” from FIG. 2A to 2B, feature reduction is performed on the first training set 216 based on the first machine learning model 218. The first machine learning model 218 is evaluated to determine which features or input information is most useful in predicting the target feature 204. This is a feature reduction that reduces the large number of features in the first training set 216 to a smaller set of relevant features 220.

A feature importance engine 222 is used to evaluate the importance of the features in the first training set 216. Feature importance refers to techniques that calculate a score for all the input features for a given model—the scores simply represent the “importance” of each feature. Feature importance learns how the final score changes if a feature is removed and if the value for a feature is increased or decreased. A higher score means that the specific feature will have a larger effect on the model that is being used to predict a certain variable. There are many techniques known to those of ordinary skill in the art for determining feature importance. One technique is Shapley feature importance and the related SHAP (Shapley Additive exPlanations) technique. SHAP (Shapley Additive exPlanations) is a game theoretic approach to explain the output of any machine learning model. It connects optimal credit allocation with local explanations using the classic Shapley values from game theory and their related extensions. Shapley feature selection is described in D Fryer et al., “Shapley values for feature selection: The good, the bad, and the axioms.” In arXiv:2102.10936, Feb. 22, 2021.

In addition to feature importance, a causal discovery engine 224 may also be used to discover causal relationships between the features in the first training set 216. The causal relationships are based on some type of ground truth and used to capture non-linear relationships between features. Causal relationships can be identified through creation of a causal graph developed from both causal discovery and inference. Causal ML is one example of a publicly-available tool that can be used for causal inference. One technique that may be used by the causal discovery engine 224 is deep end-to-end causal inference which learns a distribution over causal graphs from observational data and subsequently estimates causal quantities. This is a single flow-based non-linear additive noise model that takes in observational data and can perform both causal discovery and inference, including conditional average treatment effect (CATE) estimation. This formulation requires assumptions that the data is generated with a non-linear additive noise model (ANM) and that there are no unobserved confounders. Deep end-to-end causal inference techniques are described in T. Geffner et al., “Deep End-to-end Causal Inference,” MSR-TR-2022-1, February 2022.

Feature reduction is performed by using one or both of feature importance and causal relationships to identify and remove features that are less useful for predicting the target feature 204. Removing irrelevant or less relevant features and data increases the accuracy of machine learning models due to dimensionality reduction and reduces the computational load necessary to run the models. Identifying relevant features also facilitates interpretability. The feature importance engine 222 and the causal discovery engine 224 may be used together in multiple ways. In one implementation, only features with more than a threshold relevance and more than a threshold strength of causal relationship are retained. In other implementations, first features are analyzed by the feature importance engine 222 and then only those features with more than a threshold level of importance are evaluated by the causal discovery engine 224 for causal relationships. Alternatively, the causal discovery engine 224 may be used first to identify only those features with a causal relationship and then the features identified by the causal discovery engine 224 are provided to the feature importance engine 222.

In one implementation, a predetermined number of the most relevant features 220 are retained and all others are removed. The predetermined number may be any number of features such as 10, 20, 30, 40, 50, 60, 70, 80, 90, 100 or a different number. The number of features to retain as relevant features 220 may be determined by Principal Component Analysis. In one implementation for predicting ileal digestibility values, Principal Component Analysis identified that the top 20 features explained 95% of the model variance. Therefore, these 20 features were selected as relevant features 220. Thus, feature reduction by the feature importance engine 222 and the causal discovery engine 224 may be used to reduce the number of features in the dataset from hundreds (or more) to tens of relevant features.

A second training set 226 is created from only those features identified as relevant by the feature importance engine 222 and/or the causal discovery engine 224. The second training set 226 includes the same target feature 204 (i.e., digestibility values) as the first training set 216 but only a subset of the other features (i.e., other information 208 and physiochemical features 214).

For example, if the target feature 204 is the ileal digestibility score used to calculate DIAAS, the inventors have identified 37 relevant features 220 that are listed in the table below.

| # | Feature Name |
|---|---|
| 1 | energy (kJ/100 g) |
| 2 | dietary fiber (g/100 g) |
| 3 | fat (g/100 g) |
| 4 | ash (g/100 g) |
| 5 | total sugars (g/100 g) |
| 6 | calcium (mg/100 g) |
| 7 | phosphorus (mg/100 g) |
| 8 | magnesium (mg/100 g) |
| 9 | potassium (mg/100 g) |
| 10 | sodium (mg/100 g) |
| 11 | zinc (mg/100 g) |
| 12 | copper (mg/100 g) |
| 13 | iron (mg/100 g) |
| 14 | selenium (μg/100 g) |
| 15 | total protein (g/kg) |
| 16 | tryptophan (g/kg) |
| 17 | threonine (g/kg) |
| 18 | isoleucine (g/kg) |
| 19 | leucine (g/kg) |
| 20 | lysine (g/kg) |
| 21 | methionine (g/kg) |
| 22 | cysteine (g/kg) |
| 23 | phenylalanine (g/kg) |
| 24 | tyrosine (g/kg) |
| 25 | valine (g/kg) |
| 26 | arginine (g/kg) |
| 27 | histidine (g/kg) |
| 28 | 1^{st} identified protein family |
| 29 | 2^{nd} identified protein family |
| 30 | 3^{rd} identified protein family |
| 31 | food group |
| 32 | the pK value of a carboxyl (—COOH) group |
| 33 | average weighted atomic number or degree based on atomic number in the graph |
| 34 | entire chain composition of amino acids in intracellular proteins of thermophiles (%) |
| 35 | linker propensity from small dataset (linker length is less than six residues) |
| 36 | normalized positional residue frequency at helix termini C |
| 37 | hydration number |

The 1s t 2n d and 3r d identified protein families are the names of the most abundant protein families in the food item 202. The food group is a categorical label that identifies the food group to which the food item 202 belongs.

Returning to FIG. 2A, the protein sequence 206 is also processed by an embeddings engine 228 to generate embeddings 230. The embeddings engine 228 may use a deep learning architecture, such as a transformer model, to extract an embedding that is a fixed size vector from the protein sequence 206. Transformers are a family of neural network architectures that compute dense, context-sensitive representations for tokens which in this implementation will be the amino acids of the protein sequence 206. Use of a language model approach, such as transformers, treats the protein sequence 206 a series of tokens, or characters, like a text corpus. However, any technique that creates embeddings 230 in a latent space from a string of amino acids may be used such as, for example, a variational autoencoder (VAE).

In one implementation, the embeddings engine 228 is implemented by the pre-trained transformer-based protein Language Model (pLM) such as ProtTrans. Protein Language Models copy the concepts of Language Models from natural language processing (NLP) by using tokens (words in NLP), i.e., amino acids from protein sequences, and treating entire proteins like sentences in Language Models. The pLMs are trained in a self-supervised manner, essentially learning to predict masked amino acids (tokens) in already known sequences.

In one implementation, the embeddings 230 are created by the encoder portion of ProtTrans. The embeddings 230 can be represented as a high-dimensional vector. For example, the high-dimensional vector may have 512, 1024, or another number of embeddings. In this implementation, one vector representing multiple embeddings 230 is generated for each protein sequence 206. ProtTrans uses two auto-regressive models (Transformer-XL, XLNet) and four auto-encoder models (BERT, Albert, Electra, T5) trained on data from UniRef50 and BFD100 containing up to 393 billion amino acids. The embeddings 230 generated by ProtTrans are provided as high-dimensionality vectors. The embeddings 230 are believed to be related to physiochemical properties of the proteins. ProtTrans is described in A. Elnaggar et al., “ProtTrans: Towards Cracking the Language of Lifes Code Through Self-Supervised Deep Learning and High Performance Computing,” in IEEE Transactions on Pattern Analysis and Machine Intelligence.

Following path “B” from FIG. 2A to 2B, the embeddings 230 generated by the embeddings engine 228 are also added to the second training set 226. In an implementation, the embeddings are not included in the first training set 216. Thus, the second training set 226 includes the label for the training data (i.e., the target feature 204), the relevant features 220 which are a subset of the other features identified by one or both of feature importance and causal discovery, and the embeddings 230. The relevant features 220 may include relevant physiochemical features which are a subset of all the physiochemical features 214 that may be identified by the feature extraction engine 212. The relevant features 220 may also include relevant other information (e.g., protein information) that is a subset of the other information 208 which is available for a food item 202.

The second training set 226 is used to train a second machine learning model 232. The second machine learning model 232 may be the same as the first machine learning model 218 or it may be a different type of machine learning model. For example, the second machine learning model 232 may include a regressor, a decision tree, a random forest, XGBoost, LightGBM, or another machine learning technique. The second machine learning model 232 may use a linear or non-linear technique. Due to the selection and reduction of features used for the second training set 226 and inclusion of the embeddings, the second machine learning model 232 provides more accurate predictions for the target feature 204 than the first machine learning model 218. For example, the R 2 value for the first machine learning model 218 that uses LightGBM to predict ileal digestibility values increased from 0.87730 to 0.90165 in the second machine learning model 232 after feature selection using SHAP and addition of transformer embeddings.

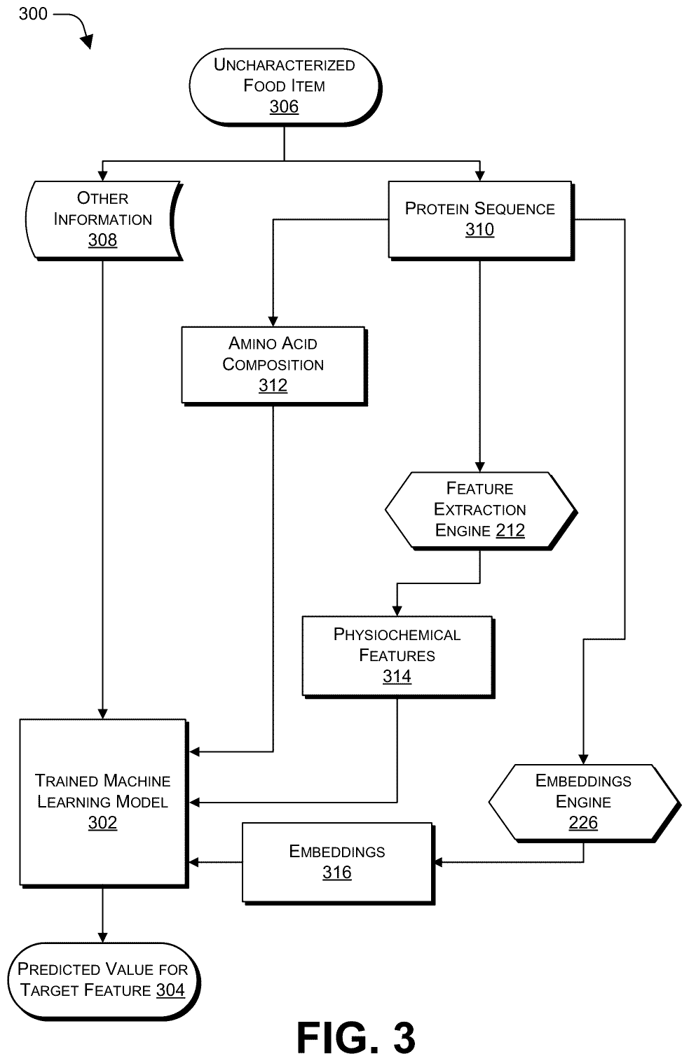

FIG. 3 shows an architecture 300 for the use of a trained machine learning model 302 to generate a predicted value for a target feature 304 of an uncharacterized food item 306. The trained machine learning model 302 may be the same as the second machine learning model 232 shown in FIG. 2. The uncharacterized food item 306 is a food item for which the value of the target feature is not known. Of course, the accuracy of this machine learning technique may be tested by using the model to analyze food items for which the value for the target feature has been determined experimentally.

Other information 308 is obtained for the uncharacterized food item 306. The other information 308 includes the same features that are in the second training set 224. For example, the other information 308 may include nutritional information and category information such as type of food item and protein family of proteins in the uncharacterized food item 306. If the target feature is digestibility, for example, the other information 308 may include energy, dietary fiber, and fat content as well as other characteristics. The other information 308 for the uncharacterized food item 306 can include only the relevant features which may be many fewer than the other information 208 used to train the first machine learning model 218 shown in FIG. 2A.

The protein sequence 310 is also obtained for one or more proteins in one or more protein families in the uncharacterized food item 306. The protein sequence 310 will generally be known and can be obtained from an existing database. However, it is also possible that the protein sequence 310 is discovered by protein sequencing or determined by analysis of a gene sequence.

The amino acid composition 312 is determined from the protein sequence 310 by the same technique used for training the machine learning model. The feature extraction engine 212 (e.g., the Protlearn tool) is used to determine physiochemical features 314. Again, only the physiochemical features that were identified as relevant by the feature importance engine 222 and/or the causal discovery engine 224 are needed. Because the physiochemical features 314 are only a subset of all the physiochemical features that could be generated by the feature extraction engine 212, there is a savings in both computational time and processing cycles.

Embeddings 316 are generated from the protein sequence 310 by the embeddings engine 226. The embeddings 316 are generated in the same way as the embeddings 316 used to train the trained machine learning model 302. For example, the embeddings engine 226 may be implemented by the ProtTrans model. The other information 308, amino acid composition 312, physiochemical features 314, and embeddings 316 are provided to the trained machine learning model 302.

The trained machine learning model 302, based on learned correlations, generates a predicted value for the target feature 304 from the input features. When the trained machine learning model 302 was trained to predict ileal digestibility coefficients, it accurately predicted the correct ileal digestibility coefficient for proteins in food items that were not in the training set with a surprisingly high 91% accuracy.

The predicted value for the target feature 304 may be used to determine another characteristic or feature of the uncharacterized food item 306. For example, if the trained machine learning model 302 predicts a value for ileal digestibility or feal digestibility, that value can be converted into a digestibility score such as PDCAAS or DIAAS using known techniques.

### Example Methods

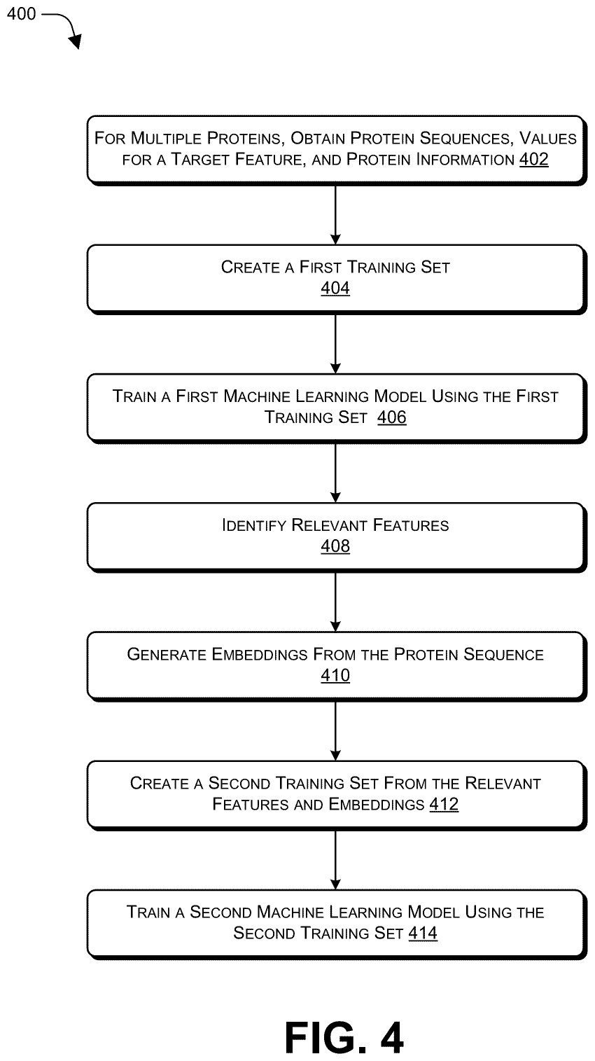

FIG. 4 illustrates example method 400 for training machine learning models according to the techniques of this disclosure. Method 400 may be implemented through the techniques shown in the middle frame 108 of FIG. 1 or the architecture 200 shown in FIGS. 2A and 2B.

At operation 402, protein sequences, values for a target feature, and other protein information is obtained for multiple proteins. The multiple proteins may be proteins identified from multiple food items. One or more proteins may be identified for each food item. The target feature is a feature or characteristic of the proteins that a machine learning model will be trained to predict. For food proteins, the target feature may be, for example, digestibility, texture, or flavor. The other protein information may be any other type of information about a protein or a food item containing the protein that is not the target feature and not derived from the protein sequence. For food proteins, the protein information may be nutritional information about the food item that contains the protein. The protein sequences may be obtained from existing databases of protein sequences. Protein sequences may also be determined from nucleic acid sequences that code for a protein.

At operation 404, a first training set is created for the data from the multiple proteins. The first training set may contain data from many hundreds, thousands, or more proteins. The first training set includes the values for the target feature, the protein information, and physiochemical features determined from the protein sequences. The physicochemical features are determined by techniques known to persons of ordinary skill in the art. A software tool such as ProtLearn may be used to determine the physicochemical features. Given the large number of proteins used to create a robust set of training data, manual determination of physiochemical features is impractical. Examples of physiochemical features include amino acid composition, pK value of carboxyl group, average weighed atomic number, degree based on atomic number in a graph, linker propensity from small dataset, normalized positional residue frequency at helix termini C, and hydration number.

The protein sequence itself may or may not be included in the first training set. The first training set may also include elements of three-dimensional protein structures derived from the protein sequence.

At operation 406, a first machine learning model is trained using the first training set. This is supervised training using labeled training data. The first machine learning model may be any type of machine learning model. For example, it may be a regressor, decision trees, or a random forest. The first machine learning model may use a gradient boosting technique such as XGBoost or LightGBM.

At operation 408, a subset of the features from the first training set that were used to train the first machine learning model are identified as relevant features. As used herein, “relevant” features are those features that have a greater effect on prediction of a value for the target feature than other features. Relevant features may be identified by comparison to a threshold value of a relevance score—features with a value above the threshold are deemed relevant. Alternatively, instead of using all features with more than a threshold relevance, only a fixed number of features (e.g., 10, 20, 30, 40, 50, or another number) with the highest relevance scores are designated as relevant. The relevant features include both features from the protein information and physiochemical features derived from the protein sequences.

In one implementation, the relevant features are identified using feature importance. Any known or later developed technique for identifying feature important may be used. For example, Shapley values may be used to determine feature importance. In one implementation, the relevant features are identified by causal relationships. Any known or later developed technique for causal discovery and inference may be used. For example, Conditional Average Treatment Effect (CATE) or Individual Treatment Effect (ITE) may be used to identify causal relationships. In some implementations, the Causal ML software tool may be used to identify causal importance.

In one implementation, both feature importance and causal relationships are used to identify relevant features. For example, only features with a feature importance that is more than a first threshold level and causal relationship strength that is more than a second threshold level are identified as relevant features. Alternatively, only those features with a causal relationship are evaluated for feature importance and features with more than a threshold level of feature importance are deemed relevant features. Thus, both Shapley values and causal relationships determined by CATE or ITE may be used to identify relevant features.

At operation 410, embeddings are created from the protein sequences. The embeddings may be generated by any technique that takes protein sequences and converts them into vectors in a latent space. In one implementation, the embeddings are created by a transformer model such as ProtTrans. The creation of embeddings is a type of unsupervised learning that can separate protein families and extract physicochemical features from the primary protein structure.

At operation 412, a second training set is created from the relevant features identified at operation 408, the embeddings generated at operation 410, and the target features. The second training set includes the embeddings which are not in the first training set. Typically, the second training set will contain fewer features than the first training. However, if most of the features from the first training set are identified as relevant features, addition of the embeddings may result in the second training set having more features than the first training set.

At operation 414, a second machine learning model is trained using the second training set. The second machine learning model may be any type of machine learning model and may be the same or different type of machine learning model as the first machine learning model. The second machine learning model is trained using standard machine learning techniques to learn a non-linear relationship between the target feature and the other features included in the second training set. Due to training on only relevant features and inclusion of the embeddings, the second machine learning model will generally provide better predictions than the first machine learning model.

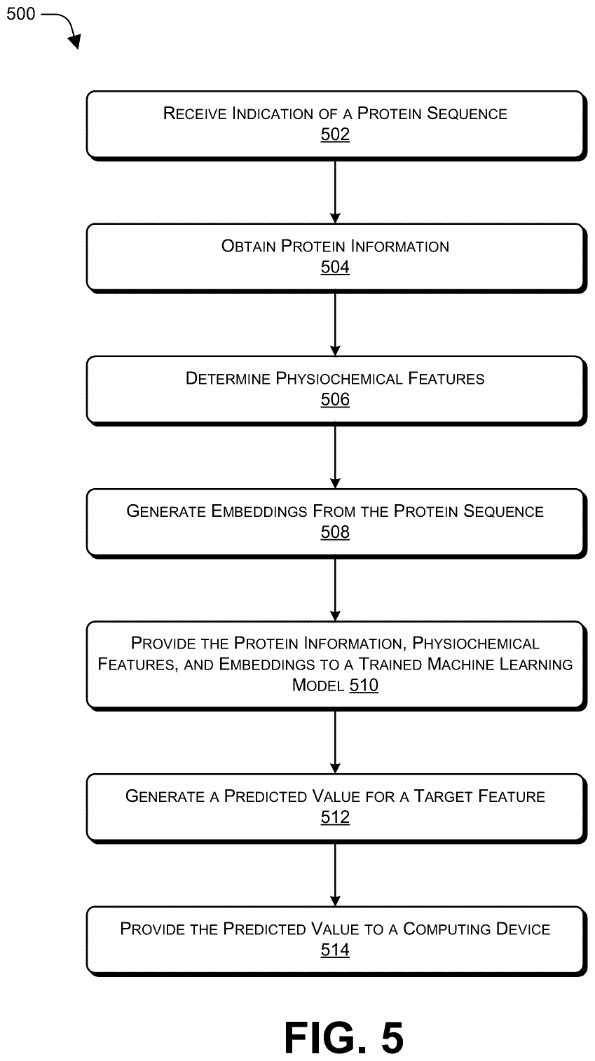

FIG. 5 illustrates example method 500 for providing a predicted value of a target feature for a protein sequence. Method 500 may be implemented through the techniques shown in the bottom frame 114 of FIG. 1 or the architecture 300 shown in FIG. 3.

At operation 502, an indication of a protein sequence is received. The indication of the protein sequence may be the protein sequence itself or other information such as identification of a food item that contains the protein. The indication may be received from a computing device such as a mobile computing device. A user may, for example, manually enter the protein sequence or provide a file such as a FASTA formatted file containing the protein sequence. Alternatively, a nucleotide sequence may be provided, and the protein sequence is determined using conventional translation rules.

At operation 504, protein information for the protein sequence is obtained. In one implementation the protein information is provided together with the protein sequence. For example, a user may provide both the protein sequence and protein information as a query. In another implementation, the protein information is obtained from a database. Thus, a user may provide only the name of the protein or protein sequence and the protein information is then retrieved from another source. In some implementations, a food item that contains the protein is also provided as part of the query. The food item may be used to look up protein information such as nutritional information. The nutritional information may include energy content, dietary fiber amount, fat quantity, ash quantity, total sugar, calcium content, phosphorus content, sodium content, zinc content, copper content, and iron content.

Although any amount of protein information may be obtained, only protein information for relevant features (e.g., as identified in operation 408 of method 400) is needed. Obtaining only a subset of all available protein information may reduce retrieval times and decrease the amount of data that needs to be transmitted over a network. It may also reduce the data entry burden on a user that is providing the protein information manually.

At operation 506, physiochemical features are determined from the protein sequence received at operation 502. The physiochemical features that are determined are those identified as relevant features in operation 408 of method 400. Although the same physiochemical features are determined for predicting a target value as for training the machine learning model, the specific techniques to determine each feature and software tools used to do so may be different. The physiochemical features may be determined by ProtLearn or another software tool. The physiochemical features may include any one or more of amino acid composition, pK value of carboxyl group, average weighed atomic number, degree based on atomic number in a graph, linker propensity from small dataset, normalized positional residue frequency at helix termini C, and hydration number.

At operation 508, embeddings are generated from the protein sequence. The embeddings are generated by the same technique used to generate the embeddings for training the machine learning model. For example, the embeddings may be generated with a transformer model such as ProtTrans. The embeddings may be represented as a vector such as a high-dimensional vector.

At operation 510, the protein information, the physiochemical features, and the embeddings are provided to a trained machine learning model. The trained machine learning model is trained on multiple proteins with known values for a target feature. One example of suitable training is that illustrated by method 400 in FIG. 4. The trained machine learning model may be any type of machine learning model. In some implementations, the machine learning model is a regressor.

At operation 512 a predicted value for the target feature for the protein sequence is generated by the trained machine learning model. The target feature may be any feature or characteristic of the protein for which the trained machine learning model is trained. For example, the target feature may be digestibility, texture, or flavor of the protein.

At operation 514, the predicted value for the target feature (or another value derived therefrom) is provided to a computing device. This may be the same computing device that supplied the indication of the protein sequence at operation 502. The predicted value for the target feature may be surfaced to a user of the computing device through an interface such as a specific application or app. If the system that maintains the machine learning model is a networked-based system or located on the “cloud,” a web-based interface may be used to present the results. A local system may use locally installed software and not a web-based interface to present the results. The final predicted value for the target feature may be surfaced by itself, together with intermediate results such as the ileal or fecal digestibility coefficient, or the intermediate results may be presented instead of the value for the target feature.

Computing Devices and Systems

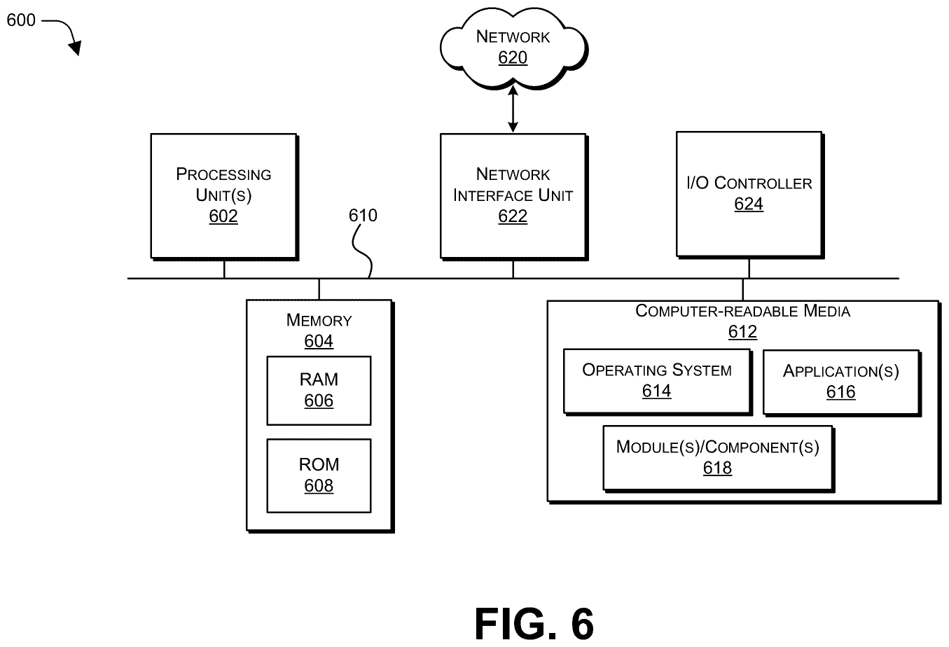

FIG. 6 shows details of an example computer architecture 600 for a device, such as a computer or a server configured as part of a local or cloud-based platform, capable of executing computer instructions (e.g., a module or a component described herein). The computer architecture 600 illustrated in FIG. 6 includes processing unit(s) 602, a memory 604, including a random-access memory 606 (“RAM”) and a read-only memory (“ROM”) 608, and a system bus 610 that couples the memory 604 to the processing unit(s) 602. The processing unit(s) 602 include one or more hardware processors and may also comprise or be part of a processing system. In various examples, the processing units 602 of the processing system are distributed. Stated another way, one processing unit 602 may be located in a first location (e.g., a rack within a datacenter) while another processing unit 602 of the processing system is located in a second location separate from the first location.

The processing unit(s) 602 can represent, for example, a CPU-type processing unit, a GPU-type processing unit, a field-programmable gate array (FPGA), another class of digital signal processor (DSP), or other hardware logic components that may, in some instances, be driven by a CPU. For example, illustrative types of hardware logic components that can be used include Application-Specific Integrated Circuits (ASICs), Application-Specific Standard Products (ASSPs), System-on-a-Chip Systems (SOCs), Complex Programmable Logic Devices (CPLDs), and the like.

A basic input/output system containing the basic routines that help to transfer information between elements within the computer architecture 600, such as during startup, is stored in the ROM 608. The computer architecture 600 further includes a computer-readable media 612 for storing an operating system 614, application(s) 616, modules/components 618, and other data described herein. The application(s) 616 and the module(s)/component(s) 618 may implement training and/or use of the machine learning models described in this disclosure.

The computer-readable media 612 is connected to processing unit(s) 602 through a storage controller connected to the bus 610. The computer-readable media 612 provides non-volatile storage for the computer architecture 600. The computer-readable media 612 may by implemented as a mass storage device, yet it should be appreciated by those skilled in the art that computer-readable media can be any available computer-readable storage medium or communications medium that can be accessed by the computer architecture 600.

Computer-readable media includes computer-readable storage media and/or communication media. Computer-readable storage media can include one or more of volatile memory, nonvolatile memory, and/or other persistent and/or auxiliary computer storage media, removable and non-removable computer storage media implemented in any method or technology for storage of information such as computer-readable instructions, data structures, program modules, or other data. Thus, computer storage media includes tangible and/or physical forms of media included in a device and/or hardware component that is part of a device or external to a device, including RAM, static random-access memory (SRAM), dynamic random-access memory (DRAM), phase-change memory (PCM), ROM, erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network-attached storage, storage area networks, hosted computer storage or any other storage memory, storage device, and/or storage medium that can be used to store and maintain information for access by a computing device.

In contrast to computer-readable storage media, communication media can embody computer-readable instructions, data structures, program modules, or other data in a modulated data signal, such as a carrier wave, or other transmission mechanism. As defined herein, computer-readable storage medium does not include communication medium. That is, computer-readable storage media does not include communications media and thus excludes media consisting solely of a modulated data signal, a carrier wave, or a propagated signal, per se.

According to various configurations, the computer architecture 600 may operate in a networked environment using logical connections to remote computers through the network 620. The computer architecture 600 may connect to the network 620 through a network interface unit 622 connected to the bus 610. An I/O controller 624 may also be connected to the bus 610 to control communication in input and output devices.

It should be appreciated that the software components described herein may, when loaded into the processing unit(s) 602 and executed, transform the processing unit(s) 602 and the overall computer architecture 600 from a general-purpose computing system into a special-purpose computing system customized to facilitate the functionality presented herein. The processing unit(s) 602 may be constructed from any number of transistors or other discrete circuit elements, which may individually or collectively assume any number of states. More specifically, the processing unit(s) 602 may operate as a finite-state machine, in response to executable instructions contained within the software modules disclosed herein. These computer-executable instructions may transform the processing unit(s) 602 by specifying how the processing unit(s) 602 transitions between states, thereby transforming the transistors or other discrete hardware elements constituting the processing unit(s) 602.

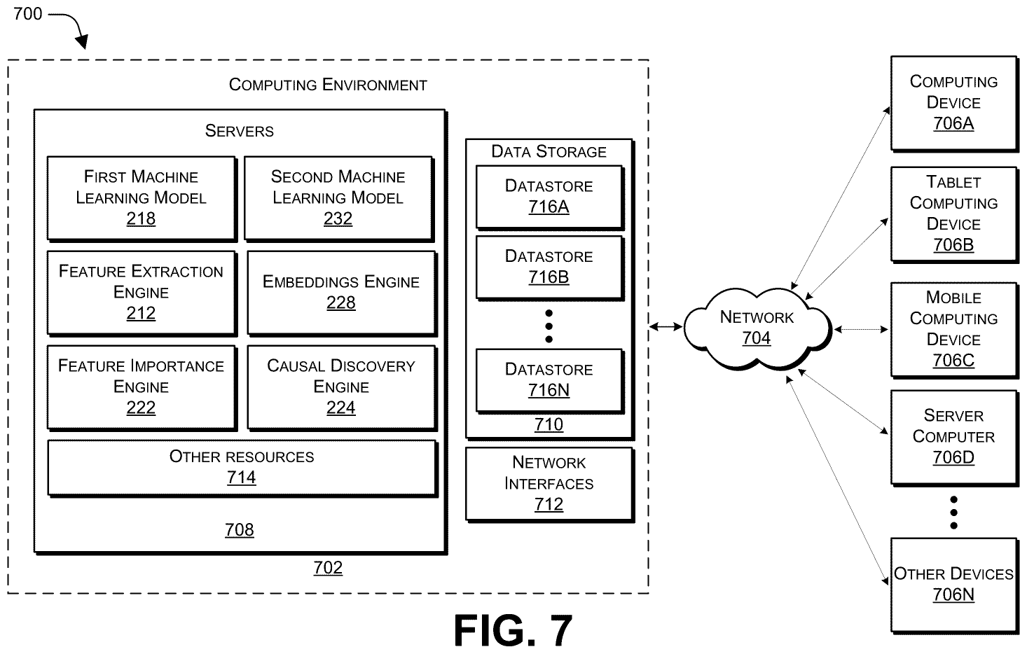

FIG. 7 depicts an illustrative distributed computing environment 700 capable of executing the components described herein. Thus, the distributed computing environment 700 illustrated in FIG. 7 can be utilized to execute any aspects of the components presented herein. Accordingly, the distributed computing environment 700 can include a computing environment 702 operating on, in communication with, or as part of a network 704. The network 704 may be the same as the network 620 shown in FIG. 6. The network 704 can include various access networks. One or more client devices 706A-706N (hereinafter referred to collectively and/or generically as “clients 706” and also referred to herein as computing devices 706) can communicate with the computing environment 702 via the network 704. The clients 706 may be any type of computing device 706A such as a laptop computer, a desktop computer, or other computing device; a slate or tablet computing device (“tablet computing device 706B”); a mobile computing device 706C such as a mobile telephone, a smart phone, or other mobile computing device; a server computer 706D; and/or other devices 706N. It should be understood that any number of clients 706 can communicate with the computing environment 702. In one implementation, a client 706 may provide an indication of a protein sequence to the computing environment 702 for the purpose of receiving a predicted value of a target feature. In one implementation, a client 706 may contain and implement the second machine learning model 232.

In various examples, the computing environment 702 includes servers 708, data storage 710, and one or more network interfaces 712. The servers 708 can host various services, virtual machines, portals, and/or other resources. In the illustrated configuration, the servers 708 host the first machine learning model 218, the second machine learning model 232, the feature extraction engine 212, the embeddings engine 226, the feature importance engine 222, and/or, the causal discovery engine 224. Each may be implemented through execution of the instructions by the one or more processing units. As shown in FIG. 7, the servers 708 also can host other services, applications, portals, and/or other resources (collectively “other resources 714”). The first machine learning model 218 can be configured to learn a first correlation between the value for the target feature, the protein information, and the physiochemical features. The second machine learning model 232 can be configured to learn a second correlation between the value for the target feature, the subset of the features, and the embeddings. The feature extraction engine 212 can be configured to determine physiochemical features from a protein sequence. The embeddings engine 228 can be configured to generate embeddings from the protein sequence. The feature importance engine 222 can be configured to identify features used to train the first machine learning model that have at least a threshold importance to the predictive power of the first machine learning model. The causal discovery engine 224 can be configured to discover causal relationships between features used to train the first machine learning model and the value for the target feature.

As mentioned above, the computing environment 702 can include the data storage 710. According to various implementations, the functionality of the data storage 710 is provided by one or more databases operating on, or in communication with, the network 704. The functionality of the data storage 710 also can be provided by one or more servers configured to host data for the computing environment 700. The data storage 710 can include, host, or provide one or more real or virtual datastores 716A-716N (hereinafter referred to collectively and/or generically as “datastores 716”). The datastores 716 are configured to host data used or created by the servers 708 and/or other data. That is, the datastores 716 also can host or store protein information such as nutritional information, known protein sequences to provide lookup functionality based on protein name, ascension number or other description, known target feature values for proteins, data structures, algorithms for execution by any of the engines provided by the servers 708, and/or other data utilized by any application program. The data storage 710 may be used to hold the first training set 216 and/or the second training set 224. The first training set 216 may include, for each of a plurality of proteins, a value for a target feature, protein information, and physiochemical features. The second training set may include, for each of the plurality of proteins, the value for a target feature, a subset of the features that have at least the threshold importance and a causal relationship to the target feature.

The computing environment 702 can communicate with, or be accessed by, the network interfaces 712. The network interfaces 712 can include various types of network hardware and software for supporting communications between two or more computing devices including the computing devices 706 and the servers 708. It should be appreciated that the network interfaces 712 also may be utilized to connect to other types of networks and/or computer systems.

It should be understood that the distributed computing environment 700 described herein can provide any aspects of the software elements described herein with any number of virtual computing resources and/or other distributed computing functionality that can be configured to execute any aspects of the software components disclosed herein. According to various implementations of the concepts and technologies disclosed herein, the distributed computing environment 700 provides the software functionality described herein as a service to the computing devices. It should be understood that the computing devices can include real or virtual machines including server computers, web servers, personal computers, mobile computing devices, smart phones, and/or other devices. As such, various configurations of the concepts and technologies disclosed herein enable any device configured to access the distributed computing environment 700 to utilize the functionality described herein for providing the techniques disclosed herein, among other aspects.

Example Nutritional Information and Physiochemical Features

Examples of nutritional information are included in the table below. The other information 208 used to create the first training set 216 may include any one or more of the features from this table.

| Name | Units | Description |
|---|---|---|
| Dry matter | % | Dry matter, difference between the total weight and the moisture |
| | | content. It is usually obtained by oven-drying, though there are |
| | | methods specific to particular products, such as silages, fats, and |
| | | molasses. |
| Crude protein | % | Crude protein, calculated as N × 6.25, where N is the nitrogen |
| | | obtained by mineralization through the Kjeldahl or Dumas |
| | | methods. |
| Crude fiber | % | Crude fiber, also known as Weende cellulose, is the insoluble |
| | | residue of an acid hydrolysis followed by an alkaline one. This |
| | | residue contains true cellulose and insoluble lignin. It is also used |
| | | to assess hair, hoof or feather residues in animal byproducts. |
| Crude fat | % | Crude fat, extracted by diethyl ether, petroleum ether or hexane. |
| | | For animal products/byproducts, gluten, potato pulp, distillery and |
| | | brewery byproducts, yeast, dairy products and bakery/biscuits |
| | | byproducts, crude fat is typically extracted after HCl hydrolysis. |
| Ash | % | Ash (mineral matter) remaining after incineration. Organic matter |
| | | is calculated as 100 - ash. |
| Insoluble ash | % | Insoluble ash, residue after incineration and treatment with |
| | | cholorhydric acid. Sometimes considered as silica. |
| NDF | % | Neutral detergent fiber, fraction of the cell walls according to Van |
| | | Soest, considered to be roughly equivalent to the sum of |
| | | hemicellulose, true cellulose and lignin. |
| ADF | % | Acid Detergent Fiber, fraction of the cell walls according to Van |
| | | Soest, considered to be roughly equivalent to the sum of true |
| | | cellulose and lignin. ADF is generally higher than crude fibre. |
| Lignin | % | Lignin, usually obtained by the Van Soest method. Acid Detergent |
| | | Lignin (ADL) is composed of both soluble and insoluble lignins. |
| Water insoluble | % | Water insoluble cell walls, according to the Carré method, used as |
| cell walls | | a predictor for the metabolizable energy of poultry feeds. This |
| | | method is described in the AFNOR norm NFV 18-111. |
| Starch | % | Starch, generally measured by polarimetry. Depending on the |
| | | method, the obtained starch value include actual starch but |
| | | possibly other carbohydrate compounds. |
| Starch, enzymatic | % | Starch obtained by an enzymatic method. |
| method | | |
| Total sugars | % | Total sugars, usually obtained by ethanol extraction, but other |
| | | methods are used depending on the product. |
| Lactose | % | Lactose, a disaccharide sugar found in milk. |
| Gross energy | kcal/kg | Gross energy, obtained by the total combustion in an calorimeter |
| (kcal) | | (usually adiabatic), expressed in kilocalories per kg. 1 kcal = |
| | | 0.0041855 MJ. |
| Gross energy (MJ) | MJ/kg | Gross energy, obtained by the total combustion in an calorimeter |
| | | (usually adiabatic), expressed in megajoules per kg. 1 MJ = |
| | | 238.92 kcal. |
| Calcium | g/kg | Calcium (Ca), a major mineral. |
| Phosphorus | g/kg | Phosphorus (P), a major mineral. |
| Phytate | g/kg | Phytate phosphorus is the organic phosphorus bound in the form |
| phosphorus | | of phytic acid in plants. It is poorly available to animals unless |
| | | freed by a phytase enzyme. |
| Magnesium | g/kg | Magnesium (Mg), a major mineral. |
| Potassium | g/kg | Potassium (K), a major mineral. |
| Sodium | g/kg | Sodium (Na), a major mineral. |
| Chlorine | g/kg | Chlorine (Cl), a major mineral. It expressed as an element, and not |
| | | as sodium chloride (NaCl). |
| Sulfur | g/kg | Sulfur (S), a major mineral. Alternative spelling: sulphur. |
| Dietary cation- | mEq/kg | Dietary cation-anion difference (DCAD) is the difference between |
| anion difference | | the Na and K cations and the Cl and S anions: DCAD = Na+ + |
| | | K+ − Cl− − S2−. DCDA is used to balance the acid-alkaline status in |
| | | ruminants. |
| Electrolyte | mEq/kg | Electrolytical balance (EB) is the difference between the Na and K |
| balance | | cations and the S anion: Na+ + K+ − Cl−. The EB is used to |
| | | balance the electrolyte status in ruminants. |
| Manganese | mg/kg | Manganese (Mn), a trace element. |
| Zinc | mg/kg | Zinc (Zn), a trace element. |
| Copper | mg/kg | Copper (Cu), a trace element. |
| Iron | mg/kg | Iron (Fe), a trace element. |
| Selenium | mg/kg | Selenium (Se), a trace element. |
| Cobalt | mg/kg | Cobalt (Co), a trace element. |
| Molybdenum | mg/kg | Molybdenum (Mo), a trace element. |
| Iodine | mg/kg | Iodine (I), a trace element. |
| C6 + C8 + C10 | g/kg | Sum of fatty acids C6 + C8 + C10. |
| fatty acids | | |
| C12:0 lauric acid | g/kg | Lauric acid C12:0, a saturated fatty acid. |
| C14:0 myristic | g/kg | Myristic acid C14:0, a saturated fatty acid. |
| acid | | |
| C16:0 palmitic | g/kg | Palmitic acid C16:0, a saturated fatty acid. |
| acid | | |
| C16:1 palmitoleic | g/kg | Palmitoleic acid C16:1, a monounsaturated omega-7 fatty acid. |
| acid | | |
| C18:0 stearic acid | g/kg | Stearic acid C18:0, a saturated fatty acid. |
| C18:1 oleic acid | g/kg | Oleic acid C18:1, a monounsaturated omega-9 fatty acid. |
| C18:2 linoleic acid | g/kg | Linoleic acid C18:2, a polyunsaturated omega-6 fatty acid. |
| C18:3 linolenic | g/kg | Linolenic acid C18:3, a polyunsaturated fatty acid. α-linolenic |
| acid | | acid (ALA) is an omega-3 fatty acid, common in many plant oils. |
| | | γ-linolenic acid (GLA) is an omega-6 fatty acid. |
| C18:4 stearidonic | g/kg | Stearidonic acid C18:4, a polyunsaturated omega-3 fatty acid. |
| acid | | |
| C20:0 arachidic | g/kg | Arachidic acid C20:0, a saturated fatty acid. |
| acid | | |
| C20:1 eicosenoic | g/kg | Eicosenoic acid C20:1, a monounsaturated fatty acid that exists in |
| acid | | three different forms: 9-eicosenoic acid (gadoleic acid), an omega- |
| | | 11 fatty acid common in fish oils, 11-eicosenoic acid (gondoic |
| | | acid), an omega-9 fatty acid characteristic of jojoba oil, and 13- |
| | | eicosenoic acid (paullinic acid), an omega-7 fatty acid. |
| C20:4 arachidonic | g/kg | Arachidonic acid C20:4, a polyunsaturated omega-6 fatty acid. |
| acid | | |
| C20:5 | g/kg | Eicosapentaenoic acid C20:5, an omega-3 polyunsaturated fatty |
| eicosapentaenoic | | acid. Other name: timnodonic acid. |
| acid | | |
| C22:0 behenic | g/kg | Behenic acid C22:0, a saturated fatty acid. Synonym: docosanoic |
| acid | | acid. |
| C22:1 erucic acid | g/kg | Erucic acid C22:1, a monounsaturated omega-9 fatty acid. |
| C22:5 | g/kg | Docosapentaenoic acid (DPA) C22:5, a polyunsaturated fatty |
| docosapentaenoic | | acid. All-cis-4,7,10,13,16-docosapentaenoic acid (osbond acid) is |
| acid | | an omega-6 fatty acid. All-cis-7,10,13,16,19-docosapentaenoic |
| | | acid (clupanodonic acid) is an omega-3 fatty acid. |
| C22:6 | g/kg | Docosahexaenoic acid (DHA) C22:6, a polyunsaturated omega-3 |
| docosahexaenoic | | fatty acid. Other name: cervonic acid. |
| acid | | |
| C24:0 lignoceric | g/kg | Lignoceric acid C24:0, a saturated fatty acid. |
| acid | | |
| Fatty acids | % | Total fatty acids, expressed either in % (as fed or DM), or in % of |
| | | crude fat. This sum may not correspond exactly to the sum of |
| | | individual fatty acids due to rounding errors and other sources of |
| | | uncertainty. |
| Vitamin A | 1000 | Vitamin A refers to a group of fat-soluble organic compounds that |
| | IU/kg | includes retinol, retinal and retinoic acid. The carotenes α- |
| | | carotene, β-carotene, γ-carotene and the xanthophyll β- |
| | | cryptoxanthin function as provitamin A in herbivores and |
| | | omnivore animals that possess the enzyme β-carotene 15,15′- |
| | | dioxygenase. Vitamin A is found in numerous animal products |
| | | whereas provitamins A are found in plants. |
| Vitamin D | 1000 | Vitamin D (D2 ergocalciferol and D3 cholecalciferol), is a fat- |
| | IU/kg | soluble organic compound (secosteroid) responsible for increasing |
| | | intestinal absorption of calcium, magnesium, and phosphate, and |
| | | multiple other biological effects. It is found in fungi, in plants (for |
| | | vitamin D2) and animals (for vitamin D3). |
| Vitamin E | mg/kg | Vitamin E refers to a group of fat-soluble organic compounds with |
| | | antioxidant properties. γ-tocopherol and α-tocopherol are the most |
| | | common forms. Vitamin E is found in a wide range of plant |
| | | products. |
| Vitamin K | mg/kg | Vitamin K is a group of structurally similar, fat-soluble organic |
| | | compounds required by animals for complete synthesis of certain |
| | | proteins that are prerequisites for blood coagulation. Vitamin K |
| | | includes two natural forms: vitamin K1 (phylloquinone, found in |
| | | plant) and vitamin K2 (menaquin, produced by microbial |
| | | synthesis). |
| Vitamin B1 | mg/kg | Vitamin B1, also known as thiamine or thiamin, is a water-soluble |
| thiamin | | organic compound needed for the metabolism of carbohydrates. It |
| | | is found in a wide range of plant and animal products. |
| Vitamin B2 | mg/kg | Vitamin B2, also known as riboflavin, is a water-soluble organic |
| riboflavin | | compound that functions as a coenzyme. It is found in numerous |
| | | plant and animal products. |
| Vitamin B6 | mg/kg | Vitamin B6 refers to a group of water-soluble organic compounds |
| pyridoxine | | that serve as coenzymes in some 100 enzyme reactions in amino |
| | | acid, glucose, and lipid metabolism. The main form of vitamin |
| | | B6 is pyridoxin. Vitamin B6 is found in a wide range of plant and |
| | | animal products. |
| Vitamin B12 | μg/kg | Vitamin B12, also known as cobalamin, is a water-soluble organic |
| | | compound that functions as a coenzyme in the synthesis of myelin |
| | | and in the formation of red blood cells. It is found in certain |
| | | animal products and it is also produced by bacterial fermentation |
| | | synthesis. |
| Niacin | mg/kg | Niacin, also known as nicotinic acid and vitamin PP, is an organic |
| | | compound that functions as a precursor of a coenzyme involved in |
| | | many anabolic and catabolic processes. Together |
| | | with nicotinamide it makes up the group known as vitamin |
| | | B3 complex. It is found in a wide range of animal and plant |
| | | products. |
| Pantothenic acid | mg/kg | Pantothenic acid, also called vitamin B5, is a water- |
| | | soluble organic compound required to synthesize coenzyme- |
| | | A (CoA), as well as to synthesize and |
| | | metabolize proteins, carbohydrates, and fats. It is found in a wide |
| | | range of plant and animal products. |
| Folic acid | mg/kg | Folic acid, also known as folate and vitamin B6, is a water-soluble |
| | | organic compound that is precursor of a coenzyme involved in the |
| | | synthesis of nucleic bases. Folic acid is found in a wide range of |
| | | plant and animal products. |
| Biotin | mg/kg | Biotin, also known as vitamin B7 and formerly known as vitamin |
| | | H or coenzyme R, is a water-soluble organic compound that |
| | | functions as a coenzyme for carboxylase enzymes. It is involved |
| | | in the synthesis of fatty acids, isoleucine, and valine, and |
| | | in gluconeogenesis. Biotin is found in a wide range of plant and |
| | | animal products. |
| Vitamin C | mg/kg | Vitamin C, also known as ascorbic acid and L-ascorbic acid, is a |
| | | water-soluble organic compound acting as a reducing agent |
| | | donating electrons to various enzymatic and a few non-enzymatic |
| | | reactions. It is found in a wide range of plant and animal products. |
| Choline | mg/kg | Choline is an organic compound, constitutive of lecithin, and |
| | | precursor of acetylcholine. Choline and its metabolites are needed |
| | | for the structural integrity of cell membranes, for signaling roles |
| | | and for synthesis pathways. Choline is found in a wide range of |
| | | plant and animal products. |
| Xanthophylls | mg/kg | Xanthophylls are a family of yellow carotenoid pigments found in |
| | | egg yolk, animal tissues and plants. |
| Viscosity, real | mL/g | Real applied viscosity (RAV) is measured by viscosimetry on an |
| applied | | aqueous extract (Carré et al., 1994). RAV (mL/g) = Log(RV)/C, |
| | | where RV = viscosity (extract)/viscosity (buffer) and C = |
| | | concentration (g/ml) of the material in the final extract. Unlike |
| | | potential applied viscosity, there is no extraction with 80% |
| | | ethanol, in order to keep the endogenous enzymes active. Real |
| | | applied viscosity values are not additive. |
| Phytase activity | IU/kg | Phytase activity in a sample. 1 unit is defined by the release of 1 |
| | | μmol per minute of inorganic phosphorus from a solution of |
| | | sodium phytate, at a known temperature and pH (Eeckhout and De |
| | | Paepe, 1994). |

Examples of physiochemical features that can be determined from a protein sequence are the table below. These features are indices included in the AAindex. The AAindex is available on the World Wide Web at genome.jp/aaindex/. The physiochemical features 214 determined by the feature extraction engine 212 may include any one or more of the features in this table.

| Feature Name | Describing Publication |
|---|---|
| alpha-CH chemical shifts | (Andersen et al., 1992) |
| Hydrophobicity index | (Argos et al., 1982) |
| Signal sequence helical potential | (Argos et al., 1982) |
| Membrane-buried preference parameters | (Argos et al., 1982) |
| Conformational parameter of inner helix | (Beghin-Dirkx, 1975) |
| Conformational parameter of beta-structure | (Beghin-Dirkx, 1975) |
| Conformational parameter of beta-turn | (Beghin-Dirkx, 1975) |
| Average flexibility indices | (Bhaskaran-Ponnuswamy, 1988) |
| Residue volume | (Bigelow, 1967) |
| Information value for accessibility, average fraction 35% | (Biou et al., 1988) |
| Information value for accessibility, average fraction 23% | (Biou et al., 1988) |
| Retention coefficient in TFA | (Browne et al., 1982) |
| Retention coefficient in HFBA | (Browne et al., 1982) |
| Transfer free energy to surface | (Bull-Breese, 1974) |
| Apparent partial specific volume | (Bull-Breese, 1974) |
| alpha-NH chemical shifts | (Bundi-Wuthrich, 1979) |
| alpha-CH chemical shifts | (Bundi-Wuthrich, 1979) |
| Spin-spin coupling constants 3JHalpha-NH | (Bundi-Wuthrich, 1979) |
| Normalized frequency of alpha-helix | (Burgess et al., 1974) |
| Normalized frequency of extended structure | (Burgess et al., 1974) |
| Steric parameter | (Charton, 1981) |
| Polarizability parameter | (Charton-Charton, 1982) |
| Free energy of solution in water, kcal/mole | (Charton-Charton, 1982) |
| The Chou-Fasman parameter of the coil conformation | (Charton-Charton, 1983) |
| A parameter defined from the residuals obtained from the | (Charton-Charton, 1983) |
| best correlation of the Chou-Fasman parameter of beta- | |
| sheet | |
| The number of atoms in the side chain labelled 1 + 1 | (Charton-Charton, 1983) |
| The number of atoms in the side chain labelled 2 + 1 | (Charton-Charton, 1983) |
| The number of atoms in the side chain labelled 3 + 1 | (Charton-Charton, 1983) |
| The number of bonds in the longest chain | (Charton-Charton, 1983) |
| A parameter of charge transfer capability | (Charton-Charton, 1983) |
| A parameter of charge transfer donor capability | (Charton-Charton, 1983) |
| Average volume of buried residue | (Chothia, 1975) |
| Residue accessible surface area in tripeptide | (Chothia, 1976) |
| Residue accessible surface area in folded protein | (Chothia, 1976) |
| Proportion of residues 95% buried | (Chothia, 1976) |
| Proportion of residues 100% buried | (Chothia, 1976) |
| Normalized frequency of beta-turn | (Chou-Fasman, 1978a) |
| Normalized frequency of alpha-helix | (Chou-Fasman, 1978b) |
| Normalized frequency of beta-sheet | (Chou-Fasman, 1978b) |
| Normalized frequency of beta-turn | (Chou-Fasman, 1978b) |
| Normalized frequency of N-terminal helix | (Chou-Fasman, 1978b) |
| Normalized frequency of C-terminal helix | (Chou-Fasman, 1978b) |
| Normalized frequency of N-terminal non helical region | (Chou-Fasman, 1978b) |
| Normalized frequency of C-terminal non helical region | (Chou-Fasman, 1978b) |
| Normalized frequency of N-terminal beta-sheet | (Chou-Fasman, 1978b) |
| Normalized frequency of C-terminal beta-sheet | (Chou-Fasman, 1978b) |
| Normalized frequency of N-terminal non beta region | (Chou-Fasman, 1978b) |
| Normalized frequency of C-terminal non beta region | (Chou-Fasman, 1978b) |
| Frequency of the 1st residue in turn | (Chou-Fasman, 1978b) |
| Frequency of the 2nd residue in turn | (Chou-Fasman, 1978b) |
| Frequency of the 3rd residue in turn | (Chou-Fasman, 1978b) |
| Frequency of the 4th residue in turn | (Chou-Fasman, 1978b) |
| Normalized frequency of the 2nd and 3rd residues in turn | (Chou-Fasman, 1978b) |
| Normalized hydrophobicity scales for alpha-proteins | (Cid et al., 1992) |
| Normalized hydrophobicity scales for beta-proteins | (Cid et al., 1992) |
| Normalized hydrophobicity scales for alpha + beta-proteins | (Cid et al., 1992) |
| Normalized hydrophobicity scales for alpha/beta-proteins | (Cid et al., 1992) |
| Normalized average hydrophobicity scales | (Cid et al., 1992) |
| Partial specific volume | (Cohn-Edsall, 1943) |
| Normalized frequency of middle helix | (Crawford et al., 1973) |
| Normalized frequency of beta-sheet | (Crawford et al., 1973) |
| Normalized frequency of turn | (Crawford et al., 1973) |
| Size | (Dawson, 1972) |
| Amino acid composition | (Dayhoff et al., 1978a) |
| Relative mutability | (Dayhoff et al., 1978b) |
| Membrane preference for cytochrome b: MPH89 | (Degli Esposti et al., 1990) |
| Average membrane preference: AMP07 | (Degli Esposti et al., 1990) |
| Consensus normalized hydrophobicity scale | (Eisenberg, 1984) |
| Solvation free energy | (Eisenberg-McLachlan, 1986) |
| Atom-based hydrophobic moment | (Eisenberg-McLachlan, 1986) |
| Direction of hydrophobic moment | (Eisenberg-McLachlan, 1986) |
| Molecular weight | (Fasman, 1976) |
| Melting point | (Fasman, 1976) |
| Optical rotation | (Fasman, 1976) |
| pK-N | (Fasman, 1976) |
| pK-C | (Fasman, 1976) |
| Hydrophobic parameter pi | (Fauchere-Pliska, 1983) |
| Graph shape index | (Fauchere et al., 1988) |
| Smoothed upsilon steric parameter | (Fauchere et al., 1988) |
| Normalized van der Waals volume | (Fauchere et al., 1988) |
| STERIMOL length of the side chain | (Fauchere et al., 1988) |
| STERIMOL minimum width of the side chain | (Fauchere et al., 1988) |
| STERIMOL maximum width of the side chain | (Fauchere et al., 1988) |
| N.m.r. chemical shift of alpha-carbon | (Fauchere et al., 1988) |
| Localized electrical effect | (Fauchere et al., 1988) |
| Number of hydrogen bond donors | (Fauchere et al., 1988) |
| Number of full nonbonding orbitals | (Fauchere et al., 1988) |
| Positive charge | (Fauchere et al., 1988) |
| Negative charge | (Fauchere et al., 1988) |
| pK-a (RCOOH) | (Fauchere et al., 1988) |
| Helix-coil equilibrium constant | (Finkelstein-Ptitsyn, 1977) |
| Helix initiation parameter at position i − 1 | (Finkelstein et al., 1991) |
| Helix initiation parameter at position i, i + 1, i + 2 | (Finkelstein et al., 1991) |
| Helix termination parameter at position j − 2, j − 1, j | (Finkelstein et al., 1991) |
| Helix termination parameter at position j + 1 | (Finkelstein et al., 1991) |
| Partition coefficient | (Garel et al., 1973) |
| Alpha-helix indices | (Geisow-Roberts, 1980) |
| Alpha-helix indices for alpha-proteins | (Geisow-Roberts, 1980) |
| Alpha-helix indices for beta-proteins | (Geisow-Roberts, 1980) |
| Alpha-helix indices for alpha/beta-proteins | (Geisow-Roberts, 1980) |
| Beta-strand indices | (Geisow-Roberts, 1980) |
| Beta-strand indices for beta-proteins | (Geisow-Roberts, 1980) |
| Beta-strand indices for alpha/beta-proteins | (Geisow-Roberts, 1980) |
| Aperiodic indices | (Geisow-Roberts, 1980) |
| Aperiodic indices for alpha-proteins | (Geisow-Roberts, 1980) |
| Aperiodic indices for beta-proteins | (Geisow-Roberts, 1980) |
| Aperiodic indices for alpha/beta-proteins | (Geisow-Roberts, 1980) |
| Hydrophobicity factor | (Goldsack-Chalifoux, 1973) |
| Residue volume | (Goldsack-Chalifoux, 1973) |
| Composition | (Grantham, 1974) |
| Polarity | (Grantham, 1974) |
| Volume | (Grantham, 1974) |
| Partition energy | (Guy, 1985) |
| Hydration number | (Hopfinger, 1971), Cited by |
| | Charton-Charton (1982) |
| Hydrophilicity value | (Hopp-Woods, 1981) |
| Heat capacity | (Hutchens, 1970) |
| Absolute entropy | (Hutchens, 1970) |
| Entropy of formation | (Hutchens, 1970) |
| Normalized relative frequency of alpha-helix | (Isogai et al., 1980) |
| Normalized relative frequency of extended structure | (Isogai et al., 1980) |
| Normalized relative frequency of bend | (Isogai et al., 1980) |
| Normalized relative frequency of bend R | (Isogai et al., 1980) |
| Normalized relative frequency of bend S | (Isogai et al., 1980) |
| Normalized relative frequency of helix end | (Isogai et al., 1980) |
| Normalized relative frequency of double bend | (Isogai et al., 1980) |
| Normalized relative frequency of coil | (Isogai et al., 1980) |
| Average accessible surface area | (Janin et al., 1978) |
| Percentage of buried residues | (Janin et al., 1978) |
| Percentage of exposed residues | (Janin et al., 1978) |
| Ratio of buried and accessible molar fractions | (Janin, 1979) |
| Transfer free energy | (Janin, 1979) |
| Hydrophobicity | (Jones, 1975) |
| pK (—COOH) | (Jones, 1975) |
| Relative frequency of occurrence | (Jones et al., 1992) |
| Relative mutability | (Jones et al., 1992) |
| Amino acid distribution | (Jukes et al., 1975) |
| Sequence frequency | (Jungck, 1978) |
| Average relative probability of helix | (Kanehisa-Tsong, 1980) |
| Average relative probability of beta-sheet | (Kanehisa-Tsong, 1980) |
| Average relative probability of inner helix | (Kanehisa-Tsong, 1980) |
| Average relative probability of inner beta-sheet | (Kanehisa-Tsong, 1980) |
| Flexibility parameter for no rigid neighbors | (Karplus-Schulz, 1985) |
| Flexibility parameter for one rigid neighbor | (Karplus-Schulz, 1985) |
| Flexibility parameter for two rigid neighbors | (Karplus-Schulz, 1985) |
| The Kerr-constant increments | (Khanarian-Moore, 1980) |
| Net charge | (Klein et al., 1984) |
| Side chain interaction parameter | (Krigbaum-Rubin, 1971) |
| Side chain interaction parameter | (Krigbaum-Komoriya, 1979) |
| Fraction of site occupied by water | (Krigbaum-Komoriya, 1979) |
| Side chain volume | (Krigbaum-Komoriya, 1979) |
| Hydropathy index | (Kyte-Doolittle, 1982) |
| Transfer free energy, CHP/water | (Lawson et al., 1984) |
| Hydrophobic parameter | (Levitt, 1976) |
| Distance between C-alpha and centroid of side chain | (Levitt, 1976) |
| Side chain angle theta (AAR) | (Levitt, 1976) |
| Side chain torsion angle phi (AAAR) | (Levitt, 1976) |
| Radius of gyration of side chain | (Levitt, 1976) |
| van der Waals parameter R0 | (Levitt, 1976) |
| van der Waals parameter epsilon | (Levitt, 1976) |
| Normalized frequency of alpha-helix, with weights | (Levitt, 1978) |
| Normalized frequency of beta-sheet, with weights | (Levitt, 1978) |
| Normalized frequency of reverse turn, with weights | (Levitt, 1978) |
| Normalized frequency of alpha-helix, unweighted | (Levitt, 1978) |
| Normalized frequency of beta-sheet, unweighted | (Levitt, 1978) |
| Normalized frequency of reverse turn, unweighted | (Levitt, 1978) |
| Frequency of occurrence in beta-bends | (Lewis et al., 1971) |
| Conformational preference for all beta-strands | (Lifson-Sander, 1979) |
| Conformational preference for parallel beta-strands | (Lifson-Sander, 1979) |
| Conformational preference for antiparallel beta-strands | (Lifson-Sander, 1979) |
| Average surrounding hydrophobicity | (Manavalan-Ponnuswamy, 1978) |
| Normalized frequency of alpha-helix | (Maxfield-Scheraga, 1976) |
| Normalized frequency of extended structure | (Maxfield-Scheraga, 1976) |
| Normalized frequency of zeta R | (Maxfield-Scheraga, 1976) |
| Normalized frequency of left-handed alpha-helix | (Maxfield-Scheraga, 1976) |
| Normalized frequency of zeta L | (Maxfield-Scheraga, 1976) |
| Normalized frequency of alpha region | (Maxfield-Scheraga, 1976) |
| Refractivity | (McMeekin et al., 1964), Cited |
| | by Jones (1975) |
| Retention coefficient in H | (Meek, 1980) |
| Retention coefficient in H | (Meek, 1980) |
| Retention coefficient in NaClO4 | (Meek-Rossetti, 1981) |
| Retention coefficient in NaH2PO4 | (Meek-Rossetti, 1981) |
| Average reduced distance for C-alpha | (Meirovitch et al., 1980) |
| Average reduced distance for side chain | (Meirovitch et al., 1980) |
| Average side chain orientation angle | (Meirovitch et al., 1980) |
| Effective partition energy | (Miyazawa-Jernigan, 1985) |
| Normalized frequency of alpha-helix | (Nagano, 1973) |
| Normalized frequency of bata-structure | (Nagano, 1973) |
| Normalized frequency of coil | (Nagano, 1973) |
| AA composition of total proteins | (Nakashima et al., 1990) |
| SD of AA composition of total proteins | (Nakashima et al., 1990) |
| AA composition of mt-proteins | (Nakashima et al., 1990) |
| Normalized composition of mt-proteins | (Nakashima et al., 1990) |
| AA composition of mt-proteins from animal | (Nakashima et al., 1990) |
| Normalized composition from animal | (Nakashima et al., 1990) |
| AA composition of mt-proteins from fungi and plant | (Nakashima et al., 1990) |
| Normalized composition from fungi and plant | (Nakashima et al., 1990) |
| AA composition of membrane proteins | (Nakashima et al., 1990) |
| Normalized composition of membrane proteins | (Nakashima et al., 1990) |
| Transmembrane regions of non-mt-proteins | (Nakashima et al., 1990) |
| Transmembrane regions of mt-proteins | (Nakashima et al., 1990) |
| Ratio of average and computed composition | (Nakashima et al., 1990) |
| AA composition of CYT of single-spanning proteins | (Nakashima-Nishikawa, 1992) |
| AA composition of CYT2 of single-spanning proteins | (Nakashima-Nishikawa, 1992) |
| AA composition of EXT of single-spanning proteins | (Nakashima-Nishikawa, 1992) |
| AA composition of EXT2 of single-spanning proteins | (Nakashima-Nishikawa, 1992) |
| AA composition of MEM of single-spanning proteins | (Nakashima-Nishikawa, 1992) |
| AA composition of CYT of multi-spanning proteins | (Nakashima-Nishikawa, 1992) |
| AA composition of EXT of multi-spanning proteins | (Nakashima-Nishikawa, 1992) |
| AA composition of MEM of multi-spanning proteins | (Nakashima-Nishikawa, 1992) |
| 8 A contact number | (Nishikawa-Ooi, 1980) |
| 14 A contact number | (Nishikawa-Ooi, 1986) |
| Transfer energy, organic solvent/water | (Nozaki-Tanford, 1971) |
| Average non-bonded energy per atom | (Oobatake-Ooi, 1977) |
| Short and medium range non-bonded energy per atom | (Oobatake-Ooi, 1977) |
| Long range non-bonded energy per atom | (Oobatake-Ooi, 1977) |
| Average non-bonded energy per residue | (Oobatake-Ooi, 1977) |
| Short and medium range non-bonded energy per residue | (Oobatake-Ooi, 1977) |
| Optimized beta-structure-coil equilibrium constant | (Oobatake et al., 1985) |
| Optimized propensity to form reverse turn | (Oobatake et al., 1985) |
| Optimized transfer energy parameter | (Oobatake et al., 1985) |
| Optimized average non-bonded energy per atom | (Oobatake et al., 1985) |
| Optimized side chain interaction parameter | (Oobatake et al., 1985) |
| Normalized frequency of alpha-helix from LG | (Palau et al., 1981) |
| Normalized frequency of alpha-helix from CF | (Palau et al., 1981) |
| Normalized frequency of beta-sheet from LG | (Palau et al., 1981) |
| Normalized frequency of beta-sheet from CF | (Palau et al., 1981) |
| Normalized frequency of turn from LG | (Palau et al., 1981) |
| Normalized frequency of turn from CF | (Palau et al., 1981) |
| Normalized frequency of alpha-helix in all-alpha class | (Palau et al., 1981) |
| Normalized frequency of alpha-helix in alpha + beta class | (Palau et al., 1981) |
| Normalized frequency of alpha-helix in alpha/beta class | (Palau et al., 1981) |
| Normalized frequency of beta-sheet in all-beta class | (Palau et al., 1981) |
| Normalized frequency of beta-sheet in alpha + beta class | (Palau et al., 1981) |
| Normalized frequency of beta-sheet in alpha/beta class | (Palau et al., 1981) |
| Normalized frequency of turn in all-alpha class | (Palau et al., 1981) |
| Normalized frequency of turn in all-beta class | (Palau et al., 1981) |
| Normalized frequency of turn in alpha + beta class | (Palau et al., 1981) |
| Normalized frequency of turn in alpha/beta class | (Palau et al., 1981) |
| HPLC parameter | (Parker et al., 1986) |
| Partition coefficient | (Pliska et al., 1981) |
| Surrounding hydrophobicity in folded form | (Ponnuswamy et al., 1980) |
| Average gain in surrounding hydrophobicity | (Ponnuswamy et al., 1980) |
| Average gain ratio in surrounding hydrophobicity | (Ponnuswamy et al., 1980) |
| Surrounding hydrophobicity in alpha-helix | (Ponnuswamy et al., 1980) |
| Surrounding hydrophobicity in beta-sheet | (Ponnuswamy et al., 1980) |
| Surrounding hydrophobicity in turn | (Ponnuswamy et al., 1980) |
| Accessibility reduction ratio | (Ponnuswamy et al., 1980) |
| Average number of surrounding residues | (Ponnuswamy et al., 1980) |
| Intercept in regression analysis | (Prabhakaran-Ponnuswamy, |
| | 1982) |
| Slope in regression analysis × 1.0E1 | (Prabhakaran-Ponnuswamy, |
| | 1982) |
| Correlation coefficient in regression analysis | (Prabhakaran-Ponnuswamy, |
| | 1982) |
| Hydrophobicity | (Prabhakaran, 1990) |
| Relative frequency in alpha-helix | (Prabhakaran, 1990) |
| Relative frequency in beta-sheet | (Prabhakaran, 1990) |
| Relative frequency in reverse-turn | (Prabhakaran, 1990) |
| Helix-coil equilibrium constant | (Ptitsyn-Finkelstein, 1983) |
| Beta-coil equilibrium constant | (Ptitsyn-Finkelstein, 1983) |
| Weights for alpha-helix at the window position of −6 | (Qian-Sejnowski, 1988) |
| Weights for alpha-helix at the window position of −5 | (Qian-Sejnowski, 1988) |
| Weights for alpha-helix at the window position of −4 | (Qian-Sejnowski, 1988) |
| Weights for alpha-helix at the window position of −3 | (Qian-Sejnowski, 1988) |
| Weights for alpha-helix at the window position of −2 | (Qian-Sejnowski, 1988) |
| Weights for alpha-helix at the window position of −1 | (Qian-Sejnowski, 1988) |
| Weights for alpha-helix at the window position of 0 | (Qian-Sejnowski, 1988) |
| Weights for alpha-helix at the window position of 1 | (Qian-Sejnowski, 1988) |
| Weights for alpha-helix at the window position of 2 | (Qian-Sejnowski, 1988) |
| Weights for alpha-helix at the window position of 3 | (Qian-Sejnowski, 1988) |
| Weights for alpha-helix at the window position of 4 | (Qian-Sejnowski, 1988) |
| Weights for alpha-helix at the window position of 5 | (Qian-Sejnowski, 1988) |
| Weights for alpha-helix at the window position of 6 | (Qian-Sejnowski, 1988) |
| Weights for beta-sheet at the window position of −6 | (Qian-Sejnowski, 1988) |
| Weights for beta-sheet at the window position of −5 | (Qian-Sejnowski, 1988) |
| Weights for beta-sheet at the window position of −4 | (Qian-Sejnowski, 1988) |
| Weights for beta-sheet at the window position of −3 | (Qian-Sejnowski, 1988) |
| Weights for beta-sheet at the window position of −2 | (Qian-Sejnowski, 1988) |
| Weights for beta-sheet at the window position of −1 | (Qian-Sejnowski, 1988) |
| Weights for beta-sheet at the window position of 0 | (Qian-Sejnowski, 1988) |
| Weights for beta-sheet at the window position of 1 | (Qian-Sejnowski, 1988) |
| Weights for beta-sheet at the window position of 2 | (Qian-Sejnowski, 1988) |
| Weights for beta-sheet at the window position of 3 | (Qian-Sejnowski, 1988) |
| Weights for beta-sheet at the window position of 4 | (Qian-Sejnowski, 1988) |
| Weights for beta-sheet at the window position of 5 | (Qian-Sejnowski, 1988) |
| Weights for beta-sheet at the window position of 6 | (Qian-Sejnowski, 1988) |
| Weights for coil at the window position of −6 | (Qian-Sejnowski, 1988) |
| Weights for coil at the window position of −5 | (Qian-Sejnowski, 1988) |
| Weights for coil at the window position of −4 | (Qian-Sejnowski, 1988) |
| Weights for coil at the window position of −3 | (Qian-Sejnowski, 1988) |
| Weights for coil at the window position of −2 | (Qian-Sejnowski, 1988) |
| Weights for coil at the window position of −1 | (Qian-Sejnowski, 1988) |
| Weights for coil at the window position of 0 | (Qian-Sejnowski, 1988) |
| Weights for coil at the window position of 1 | (Qian-Sejnowski, 1988) |
| Weights for coil at the window position of 2 | (Qian-Sejnowski, 1988) |
| Weights for coil at the window position of 3 | (Qian-Sejnowski, 1988) |
| Weights for coil at the window position of 4 | (Qian-Sejnowski, 1988) |
| Weights for coil at the window position of 5 | (Qian-Sejnowski, 1988) |
| Weights for coil at the window position of 6 | (Qian-Sejnowski, 1988) |
| Average reduced distance for C-alpha | (Rackovsky-Scheraga, 1977) |
| Average reduced distance for side chain | (Rackovsky-Scheraga, 1977) |
| Side chain orientational preference | (Rackovsky-Scheraga, 1977) |
| Average relative fractional occurrence in A0 (i) | (Rackovsky-Scheraga, 1982) |
| Average relative fractional occurrence in AR (i) | (Rackovsky-Scheraga, 1982) |
| Average relative fractional occurrence in AL (i) | (Rackovsky-Scheraga, 1982) |
| Average relative fractional occurrence in EL (i) | (Rackovsky-Scheraga, 1982) |
| Average relative fractional occurrence in E0 (i) | (Rackovsky-Scheraga, 1982) |
| Average relative fractional occurrence in ER (i) | (Rackovsky-Scheraga, 1982) |
| Average relative fractional occurrence in A0 (i-1) | (Rackovsky-Scheraga, 1982) |
| Average relative fractional occurrence in AR (i-1) | (Rackovsky-Scheraga, 1982) |
| Average relative fractional occurrence in AL (i-1) | (Rackovsky-Scheraga, 1982) |
| Average relative fractional occurrence in EL (i-1) | (Rackovsky-Scheraga, 1982) |
| Average relative fractional occurrence in E0 (i-1) | (Rackovsky-Scheraga, 1982) |
| Average relative fractional occurrence in ER (i-1) | (Rackovsky-Scheraga, 1982) |
| Value of theta (i) | (Rackovsky-Scheraga, 1982) |
| Value of theta (i-1) | (Rackovsky-Scheraga, 1982) |
| Transfer free energy from chx to wat | (Radzicka-Wolfenden, 1988) |
| Transfer free energy from oct to wat | (Radzicka-Wolfenden, 1988) |
| Transfer free energy from vap to chx | (Radzicka-Wolfenden, 1988) |
| Transfer free energy from chx to oct | (Radzicka-Wolfenden, 1988) |
| Transfer free energy from vap to oct | (Radzicka-Wolfenden, 1988) |
| Accessible surface area | (Radzicka-Wolfenden, 1988) |
| Energy transfer from out to in (95% buried) | (Radzicka-Wolfenden, 1988) |
| Mean polarity | (Radzicka-Wolfenden, 1988) |
| Relative preference value at N″ | (Richardson-Richardson, 1988) |
| Relative preference value at N′ | (Richardson-Richardson, 1988) |
| Relative preference value at N-cap | (Richardson-Richardson, 1988) |
| Relative preference value at N1 | (Richardson-Richardson, 1988) |
| Relative preference value at N2 | (Richardson-Richardson, 1988) |
| Relative preference value at N3 | (Richardson-Richardson, 1988) |
| Relative preference value at N4 | (Richardson-Richardson, 1988) |
| Relative preference value at N5 | (Richardson-Richardson, 1988) |
| Relative preference value at Mid | (Richardson-Richardson, 1988) |
| Relative preference value at C5 | (Richardson-Richardson, 1988) |
| Relative preference value at C4 | (Richardson-Richardson, 1988) |
| Relative preference value at C3 | (Richardson-Richardson, 1988) |
| Relative preference value at C2 | (Richardson-Richardson, 1988) |
| Relative preference value at C1 | (Richardson-Richardson, 1988) |
| Relative preference value at C-cap | (Richardson-Richardson, 1988) |
| Relative preference value at C′ | (Richardson-Richardson, 1988) |
| Relative preference value at C″ | (Richardson-Richardson, 1988) |
| Information measure for alpha-helix | (Robson-Suzuki, 1976) |
| Information measure for N-terminal helix | (Robson-Suzuki, 1976) |
| Information measure for middle helix | (Robson-Suzuki, 1976) |
| Information measure for C-terminal helix | (Robson-Suzuki, 1976) |
| Information measure for extended | (Robson-Suzuki, 1976) |
| Information measure for pleated-sheet | (Robson-Suzuki, 1976) |
| Information measure for extended without H-bond | (Robson-Suzuki, 1976) |
| Information measure for turn | (Robson-Suzuki, 1976) |
| Information measure for N-terminal turn | (Robson-Suzuki, 1976) |
| Information measure for middle turn | (Robson-Suzuki, 1976) |
| Information measure for C-terminal turn | (Robson-Suzuki, 1976) |
| Information measure for coil | (Robson-Suzuki, 1976) |
| Information measure for loop | (Robson-Suzuki, 1976) |
| Hydration free energy | (Robson-Osguthorpe, 1979) |
| Mean area buried on transfer | (Rose et al., 1985) |
| Mean fractional area loss | (Rose et al., 1985) |
| Side chain hydropathy, uncorrected for solvation | (Roseman, 1988) |
| Side chain hydropathy, corrected for solvation | (Roseman, 1988) |
| Loss of Side chain hydropathy by helix formation | (Roseman, 1988) |
| Transfer free energy | (Simon, 1976), Cited by Charton- |
| | Charton (1982) |
| Principal component I | (Sneath, 1966) |
| Principal component II | (Sneath, 1966) |
| Principal component III | (Sneath, 1966) |
| Principal component IV | (Sneath, 1966) |
| Zimm-Bragg parameter s at 20 C | (Sueki et al., 1984) |
| Zimm-Bragg parameter sigma × 1.0E4 | (Sueki et al., 1984) |
| Optimal matching hydrophobicity | (Sweet-Eisenberg, 1983) |
| Normalized frequency of alpha-helix | (Tanaka-Scheraga, 1977) |
| Normalized frequency of isolated helix | (Tanaka-Scheraga, 1977) |
| Normalized frequency of extended structure | (Tanaka-Scheraga, 1977) |
| Normalized frequency of chain reversal R | (Tanaka-Scheraga, 1977) |
| Normalized frequency of chain reversal S | (Tanaka-Scheraga, 1977) |
| Normalized frequency of chain reversal D | (Tanaka-Scheraga, 1977) |
| Normalized frequency of left-handed helix | (Tanaka-Scheraga, 1977) |
| Normalized frequency of zeta R | (Tanaka-Scheraga, 1977) |
| Normalized frequency of coil | (Tanaka-Scheraga, 1977) |
| Normalized frequency of chain reversal | (Tanaka-Scheraga, 1977) |
| Relative population of conformational state A | (Vasquez et al., 1983) |
| Relative population of conformational state C | (Vasquez et al., 1983) |
| Relative population of conformational state E | (Vasquez et al., 1983) |
| Electron-ion interaction potential | (Veljkovic et al., 1985) |
| Bitterness | (Venanzi, 1984) |
| Transfer free energy to lipophilic phase | (von Heijne-Blomberg, 1979) |
| Average interactions per side chain atom | (Warme-Morgan, 1978) |
| RF value in high salt chromatography | (Weber-Lacey, 1978) |
| Propensity to be buried inside | (Wertz-Scheraga, 1978) |
| Free energy change of epsilon (i) to epsilon (ex) | (Wertz-Scheraga, 1978) |
| Free energy change of alpha (Ri) to alpha (Rh) | (Wertz-Scheraga, 1978) |
| Free energy change of epsilon (i) to alpha (Rh) | (Wertz-Scheraga, 1978) |
| Polar requirement | (Woese, 1973) |
| Hydration potential | (Wolfenden et al., 1981) |
| Principal property value z1 | (Wold et al., 1987) |
| Principal property value z2 | (Wold et al., 1987) |
| Principal property value z3 | (Wold et al., 1987) |
| Unfolding Gibbs energy in water, pH7.0 | (Yutani et al., 1987) |
| Unfolding Gibbs energy in water, pH9.0 | (Yutani et al., 1987) |
| Activation Gibbs energy of unfolding, pH7.0 | (Yutani et al., 1987) |
| Activation Gibbs energy of unfolding, pH9.0 | (Yutani et al., 1987) |
| Dependence of partition coefficient on ionic strength | (Zaslavsky et al., 1982) |
| Hydrophobicity | (Zimmerman et al., 1968) |
| Bulkiness | (Zimmerman et al., 1968) |
| Polarity | (Zimmerman et al., 1968) |
| Isoelectric point | (Zimmerman et al., 1968) |
| RF rank | (Zimmerman et al., 1968) |
| Normalized positional residue frequency at helix termini | (Aurora-Rose, 1998) |
| N4′ | |
| Normalized positional residue frequency at helix termini N′″ | (Aurora-Rose, 1998) |
| Normalized positional residue frequency at helix termini N″ | (Aurora-Rose, 1998) |
| Normalized positional residue frequency at helix termini N′ | (Aurora-Rose, 1998) |
| Normalized positional residue frequency at helix termini Nc | (Aurora-Rose, 1998) |
| Normalized positional residue frequency at helix termini N1 | (Aurora-Rose, 1998) |
| Normalized positional residue frequency at helix termini N2 | (Aurora-Rose, 1998) |
| Normalized positional residue frequency at helix termini N3 | (Aurora-Rose, 1998) |
| Normalized positional residue frequency at helix termini N4 | (Aurora-Rose, 1998) |
| Normalized positional residue frequency at helix termini N5 | (Aurora-Rose, 1998) |
| Normalized positional residue frequency at helix termini C5 | (Aurora-Rose, 1998) |
| Normalized positional residue frequency at helix termini C4 | (Aurora-Rose, 1998) |
| Normalized positional residue frequency at helix termini C3 | (Aurora-Rose, 1998) |
| Normalized positional residue frequency at helix termini C2 | (Aurora-Rose, 1998) |
| Normalized positional residue frequency at helix termini C1 | (Aurora-Rose, 1998) |
| Normalized positional residue frequency at helix termini Cc | (Aurora-Rose, 1998) |
| Normalized positional residue frequency at helix termini C′ | (Aurora-Rose, 1998) |
| Normalized positional residue frequency at helix termini C″ | (Aurora-Rose, 1998) |
| Normalized positional residue frequency at helix termini C″′ | (Aurora-Rose, 1998) |
| Normalized positional residue frequency at helix termini C4′ | (Aurora-Rose, 1998) |
| Delta G values for the peptides extrapolated to 0M urea | (O'Neil-DeGrado, 1990) |
| Helix formation parameters (delta delta G) | (O'Neil-DeGrado, 1990) |
| Normalized flexibility parameters (B-values), average | (Vihinen et al., 1994) |
| Normalized flexibility parameters (B-values) for each | (Vihinen et al., 1994) |
| residue surrounded by no rigid neighbors | |
| Normalized flexibility parameters (B-values) for each | (Vihinen et al., 1994) |
| residue surrounded by one rigid neighbors | |
| Normalized flexibility parameters (B-values) for each | (Vihinen et al., 1994) |
| residue surrounded by two rigid neighbors | |
| Free energy in alpha-helical conformation | (Munoz-Serrano, 1994) |
| Free energy in alpha-helical region | (Munoz-Serrano, 1994) |
| Free energy in beta-strand conformation | (Munoz-Serrano, 1994) |
| Free energy in beta-strand region | (Munoz-Serrano, 1994) |
| Free energy in beta-strand region | (Munoz-Serrano, 1994) |
| Free energies of transfer of AcWI-X-LL peptides from | (Wimley-White, 1996) |
| bilayer interface to water | |
| Thermodynamic beta sheet propensity | (Kim-Berg, 1993) |
| Turn propensity scale for transmembrane helices | (Monne et al., 1999) |
| Alpha helix propensity of position 44 in T4 lysozyme | (Blaber et al., 1993) |
| p-Values of mesophilic proteins based on the distributions | (Parthasarathy-Murthy, 2000) |
| of B values | |
| p-Values of thermophilic proteins based on the distributions | (Parthasarathy-Murthy, 2000) |
| of B values | |
| Distribution of amino acid residues in the 18 non-redundant | (Kumar et al., 2000) |
| families of thermophilic proteins | |
| Distribution of amino acid residues in the 18 non-redundant | (Kumar et al., 2000) |
| families of mesophilic proteins | |
| Distribution of amino acid residues in the alpha-helices in | (Kumar et al., 2000) |
| thermophilic proteins | |
| Distribution of amino acid residues in the alpha-helices in | (Kumar et al., 2000) |
| mesophilic proteins | |
| Side-chain contribution to protein stability (kJ/mol) | (Takano-Yutani, 2001) |
| Propensity of amino acids within pi-helices | (Fodje-Al-Karadaghi, 2002) |
| Hydropathy scale based on self-information values in the | (Naderi-Manesh et al., 2001) |
| two-state model (5% accessibility) | |
| Hydropathy scale based on self-information values in the | (Naderi-Manesh et al., 2001) |
| two-state model (9% accessibility) | |
| Hydropathy scale based on self-information values in the | (Naderi-Manesh et al., 2001) |
| two-state model (16% accessibility) | |
| Hydropathy scale based on self-information values in the | (Naderi-Manesh et al., 2001) |
| two-state model (20% accessibility) | |
| Hydropathy scale based on self-information values in the | (Naderi-Manesh et al., 2001) |
| two-state model (25% accessibility) | |
| Hydropathy scale based on self-information values in the | (Naderi-Manesh et al., 2001) |
| two-state model (36% accessibility) | |
| Hydropathy scale based on self-information values in the | (Naderi-Manesh et al., 2001) |
| two-state model (50% accessibility) | |
| Averaged turn propensities in a transmembrane helix | (Monne et al., 1999) |
| Alpha-helix propensity derived from designed sequences | (Koehl-Levitt, 1999) |
| Beta-sheet propensity derived from designed sequences | (Koehl-Levitt, 1999) |
| Composition of amino acids in extracellular proteins | (Cedano et al., 1997) |
| (percent) | |
| Composition of amino acids in anchored proteins (percent) | (Fukuchi-Nishikawa, 2001) |
| Composition of amino acids in membrane proteins (percent) | (Fukuchi-Nishikawa, 2001) |
| Composition of amino acids in intracellular proteins | (Fukuchi-Nishikawa, 2001) |
| (percent) | |
| Composition of amino acids in nuclear proteins (percent) | (Fukuchi-Nishikawa, 2001) |
| Surface composition of amino acids in intracellular proteins | (Fukuchi-Nishikawa, 2001) |
| of thermophiles (percent) | |
| Surface composition of amino acids in intracellular proteins | (Fukuchi-Nishikawa, 2001) |
| of mesophiles (percent) | |
| Surface composition of amino acids in extracellular proteins | (Fukuchi-Nishikawa, 2001) |
| of mesophiles (percent) | |
| Surface composition of amino acids in nuclear proteins | (Fukuchi-Nishikawa, 2001) |
| Interior composition of amino acids in intracellular proteins | (Fukuchi-Nishikawa, 2001) |
| of thermophiles (percent) | |
| Interior composition of amino acids in intracellular proteins | (Fukuchi-Nishikawa, 2001) |
| of mesophiles (percent) | |
| Interior composition of amino acids in extracellular proteins | (Fukuchi-Nishikawa, 2001) |
| of mesophiles (percent) | |
| Interior composition of amino acids in nuclear proteins | (Fukuchi-Nishikawa, 2001) |
| (percent) | |
| Entire chain composition of amino acids in intracellular | (Fukuchi-Nishikawa, 2001) |
| proteins of thermophiles (percent) | |
| Entire chain composition of amino acids in intracellular | (Fukuchi-Nishikawa, 2001) |
| proteins of mesophiles (percent) | |
| Entire chain composition of amino acids in extracellular | (Fukuchi-Nishikawa, 2001) |
| proteins of mesophiles (percent) | |
| Entire chain composition of amino acids in nuclear proteins | (Fukuchi-Nishikawa, 2001) |
| (percent) | |
| Screening coefficients gamma, local | (Avbelj, 2000) |
| Screening coefficients gamma, non-local | (Avbelj, 2000) |
| Slopes tripeptide, FDPB VFF neutral | (Avbelj, 2000) |
| Slopes tripeptides, LD VFF neutral | (Avbelj, 2000) |
| Slopes tripeptide, FDPB VFF noside | (Avbelj, 2000) |
| Slopes tripeptide FDPB VFF all | (Avbelj, 2000) |
| Slopes tripeptide FDPB PARSE neutral | (Avbelj, 2000) |
| Slopes dekapeptide, FDPB VFF neutral | (Avbelj, 2000) |
| Slopes proteins, FDPB VFF neutral | (Avbelj, 2000) |
| Side-chain conformation by gaussian evolutionary method | (Yang et al., 2002) |
| Amphiphilicity index | (Mitaku et al., 2002) |
| Volumes including the crystallographic waters using the | (Tsai et al., 1999) |
| ProtOr | |
| Volumes not including the crystallographic waters using the | (Tsai et al., 1999) |
| ProtOr | |
| Electron-ion interaction potential values | (Cosic, 1994) |
| Hydrophobicity scales | (Ponnuswamy, 1993) |
| Hydrophobicity coefficient in RP-HPLC, C18 with | (Wilce et al. 1995) |
| 0.1% TFA/MeCN/H2O | |
| Hydrophobicity coefficient in Rwith 0.1% TFA/MeCN/H2O | (Wilce et al. 1995) |
| Hydrophobicity coefficient in Rwith 0.1% TFA/MeCN/H2O | (Wilce et al. 1995) |
| Hydrophobicity coefficient in RP-HPLC, C18 with | (Wilce et al. 1995) |
| 0.1% TFA/2-PrOH/MeCN/H2O | |
| Hydrophilicity scale | (Kuhn et al., 1995) |
| Retention coefficient at pH 2 | (Guo et al., 1986) |
| Modified Kyte-Doolittle hydrophobicity scale | (Juretic et al., 1998) |
| Interactivity scale obtained from the contact matrix | (Bastolla et al., 2005) |
| Interactivity scale obtained by maximizing the mean of | (Bastolla et al., 2005) |
| correlation coefficient over single-domain globular proteins | |
| Interactivity scale obtained by maximizing the mean of | (Bastolla et al., 2005) |
| correlation coefficient over pairs of sequences sharing the | |
| TIM barrel fold | |
| Linker propensity index | (Suyama-Ohara, 2003) |
| Knowledge-based membrane-propensity scale from | (Punta-Maritan, 2003) |
| 1D_Helix in MPtopo databases | |
| Knowledge-based membrane-propensity scale from | (Punta-Maritan, 2003) |
| 3D_Helix in MPtopo databases | |
| Linker propensity from all dataset | (George-Heringa, 2003) |
| Linker propensity from 1-linker dataset | (George-Heringa, 2003) |
| Linker propensity from 2-linker dataset | (George-Heringa, 2003) |
| Linker propensity from 3-linker dataset | (George-Heringa, 2003) |
| Linker propensity from small dataset (linker length is less | (George-Heringa, 2003) |
| than six residues) | |
| Linker propensity from medium dataset (linker length is | (George-Heringa, 2003) |
| between six and 14 residues) | |
| Linker propensity from long dataset (linker length is greater | (George-Heringa, 2003) |
| than 14 residues) | |
| Linker propensity from helical (annotated by DSSP) dataset | (George-Heringa, 2003) |
| Linker propensity from non-helical (annotated by DSSP) | (George-Heringa, 2003) |
| dataset | |
| The stability scale from the knowledge-based atom-atom | (Zhou-Zhou, 2004) |
| potential | |
| The relative stability scale extracted from mutation | (Zhou-Zhou, 2004) |
| experiments | |
| Buriability | (Zhou-Zhou, 2004) |
| Linker index | (Bae et al., 2005) |
| Mean volumes of residues buried in protein interiors | (Harpaz et al., 1994) |
| Average volumes of residues | (Pontius et al., 1996) |
| Hydrostatic pressure asymmetry index, PAI | (Di Giulio, 2005) |
| Hydrophobicity index | (Wolfenden et al., 1979) |
| Average internal preferences | (Olsen, 1980) |
| Hydrophobicity-related index | (Kidera et al., 1985) |
| Apparent partition energies calculated from Wertz-Scheraga | (Guy, 1985) |
| index | |
| Apparent partition energies calculated from Robson- | (Guy, 1985) |
| Osguthorpe index | |
| Apparent partition energies calculated from Janin index | (Guy, 1985) |
| Apparent partition energies calculated from Chothia index | (Guy, 1985) |
| Hydropathies of amino acid side chains, neutral form | (Roseman, 1988) |
| Hydropathies of amino acid side chains, pi-values in pH 7.0 | (Roseman, 1988) |
| Weights from the IFH scale | (Jacobs-White, 1989) |
| Hydrophobicity index, 3.0 pH | (Cowan-Whittaker, 1990) |
| Scaled side chain hydrophobicity values | (Black-Mould, 1991) |
| Hydrophobicity scale from native protein structures | (Casari-Sippl, 1992) |
| NNEIG index | (Cornette et al., 1987) |
| SWEIG index | (Cornette et al., 1987) |
| PRIFT index | (Cornette et al., 1987) |
| PRILS index | (Cornette et al., 1987) |
| ALTFT index | (Cornette et al., 1987) |
| ALTLS index | (Cornette et al., 1987) |
| TOTFT index | (Cornette et al., 1987) |
| TOTLS index | (Cornette et al., 1987) |
| Relative partition energies derived by the Bethe | (Miyazawa-Jernigan, 1999) |
| approximation | |
| Optimized relative partition energies - method A | (Miyazawa-Jernigan, 1999) |
| Optimized relative partition energies - method B | (Miyazawa-Jernigan, 1999) |
| Optimized relative partition energies - method C | (Miyazawa-Jernigan, 1999) |
| Optimized relative partition energies - method D | (Miyazawa-Jernigan, 1999) |
| Hydrophobicity index | (Engelman et al., 1986) |
| Hydrophobicity index | (Fasman, 1989) |
| Number of vertices (order of the graph) | (Karkbara-Knisley, 2016) |
| Number of edges (size of the graph) | (Karkbara-Knisley, 2016) |
| Total weighted degree of the graph (obtained by adding all | (Karkbara-Knisley, 2016) |
| the weights of all the vertices) | |
| Weighted domination number | (Karkbara-Knisley, 2016) |
| Average eccentricity | (Karkbara-Knisley, 2016) |
| Radius (minimum eccentricity) | (Karkbara-Knisley, 2016) |
| Diameter (maximum eccentricity) | (Karkbara-Knisley, 2016) |
| Average weighted degree (total degree, divided by the | (Karkbara-Knisley, 2016) |
| number of vertices) | |
| Maximum eigenvalue of the weighted Laplacian matrix of | (Karkbara-Knisley, 2016) |
| the graph | |
| Minimum eigenvalue of the weighted Laplacian matrix of | (Karkbara-Knisley, 2016) |
| the graph | |
| Average eigenvalue of the Laplacian matrix of the graph | (Karkbara-Knisley, 2016) |
| Second smallest eigenvalue of the Laplacian matrix of the | (Karkbara-Knisley, 2016) |
| graph | |
| Weighted domination number using the atomic number | (Karkbara-Knisley, 2016) |
| Average weighted eccentricity based on the atomic number | (Karkbara-Knisley, 2016) |
| Weighted radius based on the atomic number (minimum | (Karkbara-Knisley, 2016) |
| eccentricity) | |
| Weighted diameter based on the atomic number (maximum | (Karkbara-Knisley, 2016) |
| eccentricity) | |
| Total weighted atomic number of the graph (obtained by | (Karkbara-Knisley, 2016) |
| summing all the atomic number of each of the vertices in the | |
| graph) | |
| Average weighted atomic number or degree based on atomic | (Karkbara-Knisley, 2016) |
| number in the graph | |
| Weighted maximum eigenvalue based on the atomic | (Karkbara-Knisley, 2016) |
| numbers | |
| Weighted minimum eigenvalue based on the atomic | (Karkbara-Knisley, 2016) |
| numbers | |
| Weighted average eigenvalue based on the atomic numbers | (Karkbara-Knisley, 2016) |
| Weighted second smallest eigenvalue of the weighted | (Karkbara-Knisley, 2016) |
| Laplacian matrix | |

### Illustrative Embodiments

The following clauses described multiple possible embodiments for implementing the features described in this disclosure. The various embodiments described herein are not limiting nor is every feature from any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Clause 1. A method comprising: receiving an indication of a protein sequence (310); obtaining protein information (308) for the protein sequence; determining physiochemical features (314) from the protein sequence; generating embeddings (316) from the protein sequence; providing the protein information, the physiochemical features, and the embeddings to a trained machine learning model (302) that is trained on a plurality of proteins with known values for a target feature; and generating, by the trained machine learning model, a predicted value of the target feature (304) for the protein sequence.

Clause 2. The method of clause 1, further comprising, wherein the indication of the protein sequence is received from a computing device, providing the predicted value of the target feature to the computing device.

Clause 3. The method of any of clauses 1 to 2, wherein the protein information comprises nutritional information of a food item that contains a protein with the protein sequence.

Clause 4. The method of clause 3, wherein the nutritional information comprises at least one of energy content, dietary fiber amount, fat quantity, ash quantity, total sugar, calcium content, phosphorus content, sodium content, zinc content, copper content, or iron content.

Clause 5. The method of clause 3, wherein the nutritional information comprises energy content, dietary fiber amount, fat quantity, ash quantity, total sugar, calcium content, phosphorus content, sodium content, zinc content, copper content, and iron content.

Clause 6. The method of any of clauses 1 to 5, wherein the physiochemical features comprise at least one of amino acid composition, pK value of carboxyl group, average weighed atomic number, degree based on atomic number in a graph, linker propensity from small dataset, normalized positional residue frequency at helix termini C, or hydration number.

Clause 7. The method of any of clauses 1 to 5, wherein the physiochemical features comprise amino acid composition, pK value of carboxyl group, average weighed atomic number, degree based on atomic number in a graph, linker propensity from small dataset, normalized positional residue frequency at helix termini C, and hydration number.

Clause 8. The method of any of clauses 1 to 7, wherein the embeddings are created by a transformer model.

Clause 9. The method of any of clauses 1 to 8, wherein the trained machine learning model is a regressor.

Clause 10. The method of any of clauses 1 to 9, wherein the target feature is digestibility, texture, or flavor.

Clause 11. Computer-readable storage media containing instructions that, when executed by one or more processing units, cause a computing device to implement the method of any of clauses 1 to 10.

Clause 12. A method comprising: for each of a plurality of proteins (202), obtaining a protein sequence (206), a value for a target feature (204), and protein information (208); creating a first training set (216) from physiochemical features (214) determined from the protein sequence, the value for the target feature, and the protein information; training a first machine learning model (218) using the first training set; identifying a subset of features used to train the first machine learning model as relevant features (220); generating embeddings (230) from the protein sequence; creating a second training set (226) from the relevant features and the embeddings; and training a second machine learning model (232) with the second training set.

Clause 13. The method of clause 12, wherein the target feature is digestibility, texture, or flavor.

Clause 14. The method of any of clauses 12 to 13, wherein the protein information comprises nutritional information of food items that respectively contain one or more of the plurality of proteins.

Clause 15. The method of any of clauses 12 to 14, wherein the physiochemical features comprise any one of amino acid composition, pK value of carboxyl group, average weighed atomic number, degree based on atomic number in a graph, linker propensity from small dataset, normalized positional residue frequency at helix termini C, or hydration number.

Clause 16. The method of any of clauses 12 to 14, wherein the physiochemical features comprise amino acid composition, pK value of carboxyl group, average weighed atomic number, degree based on atomic number in a graph, linker propensity from small dataset, normalized positional residue frequency at helix termini C, and hydration number.

Clause 17. The method of any of clauses 12 to 16, wherein the first machine learning model comprises decision trees, random forest, or gradient boosting.

Clause 18. The method of any of clauses 12 to 17, wherein identifying the subset of features that are the relevant features uses feature importance or causal relationships.

Clause 19. The method of any of clauses 12 to 17, wherein identifying the subset of features that are the relevant features uses feature importance and causal relationships.

Clause 20. The method of clause 18, wherein identifying the subset of features that are the relevant features uses feature importance determined by Shapley values or causal relationships determined by Conditional Average Treatment Effect (CATE) or Individual Treatment Effect (ITE).

Clause 21. The method of clause 19, wherein identifying the subset of features that are the relevant features uses feature importance determined by Shapley values and causal relationships determined by Conditional Average Treatment Effect (CATE) or Individual Treatment Effect (ITE).

Clause 22. The method of any of clauses 12 to 21, wherein the embeddings are generated by a transformer model.

Clause 23. The method of any of clauses 12 to 22, wherein the second machine learning model is the same as the first machine learning model.

Clause 24. The method of any of clauses 12 to 22, wherein the second machine learning model is different than the first machine learning model.

Clause 25. The method of any of clauses 12 to 24, further comprising: receiving an indication of an uncharacterized protein; obtaining relevant protein information for the uncharacterized protein; determining relevant physiochemical features from the sequence of the uncharacterized protein; generating embeddings from the uncharacterized protein; providing the relevant protein information, the relevant physiochemical features, and the embeddings to the second machine learning model; and generating, by the second machine learning model, a predicted value of the target feature for the uncharacterized protein.

Clause 26. Computer-readable storage media containing instructions that, when executed by one or more processing units, cause a computing device to implement the method of any of clauses 12 to 25.

Clause 27. A system comprising: one or more processing units (602); computer-readable media (612) storing instructions; a feature extraction engine (212), implemented through execution of the instructions by the one or more processing units, configured to determine physiochemical features (214) from a protein sequence; a first training set (216) comprising, for each of a plurality of proteins, a value for a target feature (204), protein information (208), and the physiochemical features; a first machine learning model (218), implemented through execution of the instructions by the one or more processing units, configured to learn a first correlation between the value for the target feature, the protein information, and the physiochemical features; an embeddings engine (228), implemented through execution of the instructions by the one or more processing units, configured to generate embeddings (230) from the protein sequence; a feature importance engine (222), implemented through execution of the instructions by the one or more processing units, configured to identify features used to train the first machine learning model that have at least a threshold importance to the predictive power of the first machine learning model; a second training set (226) comprising, for each of the plurality of proteins, the value for a target feature, a subset of the features that have at least the threshold importance and a causal relationship to the target feature; and a second machine learning model (232), implemented through execution of the instructions by the one or more processing units, configured to learn a second correlation between the value for the target feature, the subset of the features, and the embeddings.

Clause 28. The system of clause 27, further comprising a causal discovery engine (224), implemented through execution of the instructions by the one or more processing units, configured to discover causal relationships between features used to train the first machine learning model and the value for the target feature.

Clause 29. The system of any of clauses 27 to 28, further comprising a network interface configured to receive an indication of an uncharacterized protein from a computing device and return to the computing device a predicted value for the target feature, the predicted value determined by providing the uncharacterized protein to the second machine learning model.

### CONCLUSION

While certain example embodiments have been described, including the best mode known to the inventors for carrying out the invention, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. Skilled artisans will know how to employ such variations as appropriate, and the embodiments disclosed herein may be practiced otherwise than specifically described. The accompanying claims and their equivalents are intended to cover such forms or modifications as would fall within the scope and spirit of certain of the inventions disclosed herein.

The terms “a,” “an,” “the” and similar referents used in the context of describing the invention are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context. The terms “portion,” “part,” or similar referents are to be construed as meaning at least a portion or part of the whole including up to the entire noun referenced.

It should be appreciated that any reference to “first,” “second,” etc. elements within the Summary and/or Detailed Description is not intended to and should not be construed to necessarily correspond to any reference of “first,” “second,” etc. elements of the claims. Rather, any use of “first” and “second” within the Summary, Detailed Description, and/or claims may be used to distinguish between two different instances of the same element (e.g., two different sensors).

In closing, although the various configurations have been described in language specific to structural features and/or methodological acts, it is to be understood that the subject matter defined in the appended representations is not necessarily limited to the specific features or acts described. Rather, the specific features and acts are disclosed as example forms of implementing the claimed subject matter.

Furthermore, references have been made to publications, patents and/or patent applications throughout this specification. Each of the cited references is individually incorporated herein by reference for its particular cited teachings as well as for all that it discloses.

1. A method comprising:
receiving an indication of a protein sequence;
obtaining other information for the protein sequence;
determining physiochemical features from the protein sequence;
generating embeddings from the protein sequence;
providing the other information, the physiochemical features, and the embeddings to a trained machine learning model that is trained on a plurality of proteins with known values for a target feature; and
generating, by the trained machine learning model, a predicted value of the target feature for the protein sequence.

2. The method of claim 1, further comprising, wherein the indication of the protein sequence is received from a computing device, providing the predicted value of the target feature to the computing device.

3. The method of claim 1, wherein the other information comprises nutritional information of a food item that contains a protein with the protein sequence.

4. The method of claim 3, wherein the nutritional information comprises energy content, dietary fiber amount, fat quantity, ash quantity, total sugar, calcium content, phosphorus content, sodium content, zinc content, copper content, and iron content.

5. The method of claim 1, wherein the physiochemical features comprise at least one of amino acid composition, pK value of carboxyl group, average weighed atomic number, degree based on atomic number in a graph, linker propensity from small dataset, normalized positional residue frequency at helix termini C, or hydration number.

6. The method of claim 1, wherein the embeddings are created by a transformer model.

7. The method of claim 1, wherein the trained machine learning model is a regressor.

8. The method of claim 1, wherein the target feature is digestibility, texture, or flavor.

9. A method comprising:
for each of a plurality of proteins, obtaining a protein sequence, a value for a target feature, and other information;
creating a first training set from physiochemical features determined from the protein sequence, the value for the target feature, and the other information;
training a first machine learning model using the first training set;
identifying a subset of features used to train the first machine learning model as relevant features;
generating embeddings from the protein sequence;
creating a second training set from the relevant features and the embeddings; and
training a second machine learning model with the second training set.

10. The method of claim 9, wherein the target feature is digestibility, texture, or flavor.

11. The method of claim 9, wherein the other information comprises nutritional information of food items that respectively contain one or more of the plurality of proteins.

12. The method of claim 9, wherein the physiochemical features comprise amino acid composition, pK value of carboxyl group, average weighed atomic number, degree based on atomic number in a graph, linker propensity from small dataset, normalized positional residue frequency at helix termini C, and hydration number.

13. The method of claim 9, wherein the first machine learning model comprises decision trees, random forest, or gradient boosting.

14. The method of claim 9, wherein identifying the subset of features that are the relevant features uses feature importance or causal relationships.

15. The method of claim 14, wherein identifying the subset of features that are the relevant features uses feature importance determined by Shapley values or causal relationships determined by Conditional Average Treatment Effect (CATE) or Individual Treatment Effect (ITE).

16. The method of claim 9, wherein the embeddings are generated by a transformer model.

17. The method of claim 9, wherein the second machine learning model is the same as the first machine learning model.

18. The method of claim 9, further comprising:
receiving an indication of an uncharacterized protein;
obtaining relevant other information for the uncharacterized protein;
determining relevant physiochemical features from the sequence of the uncharacterized protein;
generating embeddings from the uncharacterized protein;
providing the relevant other information, the relevant physiochemical features, and the embeddings to the second machine learning model; and
generating, by the second machine learning model, a predicted value of the target feature for the uncharacterized protein.

19. A system comprising:
one or more processing units;
computer-readable media storing instructions;
a feature extraction engine, implemented through execution of the instructions by the one or more processing units, configured to determine physiochemical features from a protein sequence;
a first training set comprising, for each of a plurality of proteins, a value for a target feature, other information, and the physiochemical features;
a first machine learning model, implemented through execution of the instructions by the one or more processing units, configured to learn a first correlation between the value for the target feature, the other information, and the physiochemical features;
an embeddings engine, implemented through execution of the instructions by the one or more processing units, configured to generate embeddings from the protein sequence;
a feature importance engine, implemented through execution of the instructions by the one or more processing units, configured to identify features used to train the first machine learning model that have at least a threshold importance to the predictive power of the first machine learning model;
a second training set comprising, for each of the plurality of proteins, the value for a target feature, a subset of the features that have at least the threshold importance and a causal relationship to the target feature; and
a second machine learning model, implemented through execution of the instructions by the one or more processing units, configured to learn a second correlation between the value for the target feature, the subset of the features, and the embeddings.

20. The system of claim 19, further comprising a network interface configured to receive an indication of an uncharacterized protein from a computing device and return to the computing device a predicted value for the target feature, the predicted value determined by providing the uncharacterized protein to the second machine learning model.
